(19) United States
(12) Patent Application Publication
(10) Pub. No.: US 2025/0032000 A1
(43) Pub. Date: Jan. 30, 2025

(54) FALL PROBABILITY ESTIMATION DEVICE, FALL PROBABILITY ESTIMATION SYSTEM, FALL PROBABILITY ESTIMATION METHOD, AND RECORDING MEDIUM

(71) Applicant: NEC Corporation, Minato-ku, Tokyo (JP)

(72) Inventors: Chenhui HUANG, Tokyo (JP); Fumiyuki NIHEY, Tokyo (JP); Zhenwei WANG, Tokyo (JP); Hiroshi KAJITANI, Tokyo (JP); Yoshitaka NOZAKI, Tokyo (JP); Kenichiro FUKUSHI, Tokyo (JP)

(73) Assignee: NEC Corporation, Minato-ku, Tokyo (JP)

(21) Appl. No.: 18/716,597

(22) PCT Filed: Dec. 27, 2021
(86) PCT No.: PCT/JP2021/048563

§ 371 Date: Jun. 5, 2024

(51) Int. Cl. A61B 5/11

(52) U.S. Cl. CPC A61B 5/1117

(57) ABSTRACT

Provided is a fall probability estimation device that includes a data acquisition unit that acquires feature amount data including a feature amount extracted from sensor data regarding a motion of a foot of a user and used for estimation of fall probability of the user, a storage unit that stores an estimation model that outputs a fall probability index according to an input of the feature amount data, an estimation unit that inputs the acquired feature amount data into the estimation model and estimates the fall probability of the user according to the fall probability index output from the estimation model, and an output unit that outputs information regarding the estimated fall probability of the user.

### TECHNICAL FIELD

The present disclosure relates to a fall probability estimation device and the like for estimating fall probability using sensor data regarding a motion of a foot.

### BACKGROUND ART

With increasing interest in healthcare, services for providing information according to features (also referred to as gait) included in a gait pattern have attracted attention. For example, a technique for analyzing a gait based on sensor data measured by a sensor mounted on footwear such as shoes has been developed. In time-series data of the sensor data, a feature of a gait event related to a physical condition (also referred to as a gait event) appears. For example, when the fall probability, which is one of the indices of the fall risk, can be estimated based on the feature amount extracted from the sensor data, unexpected falling or the like may be avoided.

PTL 1 discloses a device that detects an abnormality of a foot based on features of gait of a walker. The device of PTL 1 extracts a characteristic gait feature amount in gait of a walker wearing footwear by using data acquired from a sensor installed in the footwear. The device of PTL 1 detects an abnormality of the walker walking while wearing footwear based on the extracted gait feature amount. For example, the device of PTL 1 extracts a characteristic part regarding hallux valgus from gait waveform data for one gait cycle. The device of PTL 1 estimates the progression state of hallux valgus using the extracted gait feature amount of the characteristic part.

PTL 2 discloses a system for evaluating a fall risk of a management target person who is an elderly person based on a photographed image in everyday life. The system of PTL 2 authenticates the management target person photographed by a stereo camera that outputs a two-dimensional image and three-dimensional information. The system of PTL 2 tracks the authenticated management target person and calculates the feature amount of gait of the management target person. The system of PTL 2 calculates a fall index value of the management target person based on integrated data obtained by integrating the obtained data. The system of PTL 2 evaluates the fall risk of the management target person according to the relationship between the calculated fall index value and a threshold.

### CITATION LIST

### Patent Literature

PTL 1: WO 2021/140658 A1
PTL 2: WO 2021/186655 A1

### SUMMARY OF INVENTION

### Technical Problem

In the method of PTL 1, the progression state of hallux valgus is estimated using the gait feature amount of the characteristic part extracted from the data acquired from the sensor installed in the footwear. PTL 1 does not disclose that the fall probability is estimated using the gait feature amount of the characteristic part extracted from the data acquired from the sensor installed in the footwear.

In the method of PTL 2, the fall risk of the management target person is evaluated using the two-dimensional image and the three-dimensional information output from the stereo camera. In the method of PTL 2, the fall risk of the management target person can be evaluated in an environment where the image can be captured by the stereo camera. However, the method of PTL 2 cannot appropriately estimate the fall probability in an environment that cannot be covered by a stereo camera, such as a place with many obstacles or outdoors.

An object of the present disclosure is to provide a fall probability estimation device and the like capable of appropriately estimating fall probability in everyday life.

### Solution to Problem

A fall probability estimation device according to one aspect of the present disclosure includes a data acquisition unit that acquires feature amount data including a feature amount extracted from sensor data regarding a motion of a foot of a user and used for estimation of fall probability of the user, a storage unit that stores an estimation model that outputs a fall probability index according to an input of the feature amount data, an estimation unit that inputs the acquired feature amount data into the estimation model and estimates the fall probability of the user according to the fall probability index output from the estimation model, and an output unit that outputs information regarding the estimated fall probability of the user.

A fall probability estimation method according to one aspect of the present disclosure includes, acquiring feature amount data including a feature amount extracted from sensor data regarding a motion of a foot of a user and used for estimation of fall probability of the user, inputting the acquired feature amount data to an estimation model that outputs a fall probability index according to an input of the feature amount data, estimating the fall probability of the user according to the fall probability index output from the estimation model, and outputting information regarding the estimated fall probability of the user.

A program according to one aspect of the present disclosure causes a computer to execute, processing of acquiring feature amount data including a feature amount extracted from sensor data regarding a motion of a foot of a user and used for estimation of fall probability of the user, processing of inputting the acquired feature amount data to an estimation model that outputs a fall probability index according to an input of the feature amount data, processing of estimating the fall probability of the user according to the fall probability index output from the estimation model, and processing of outputting information regarding the estimated fall probability of the user.

### Advantageous Effects of Invention

According to the present disclosure, it is possible to provide a fall probability estimation device and the like capable of appropriately estimating fall probability in everyday life.

### BRIEF DESCRIPTION OF DRAWINGS

FIG. 1 is a block diagram illustrating an example of a configuration of a fall probability estimation system according to a first example embodiment.

FIG. 2 is a block diagram illustrating an example of a configuration of a gait measurement device included in the fall probability estimation system according to the first example embodiment.

FIG. 3 is a conceptual diagram illustrating an arrangement example of the gait measurement device according to the first example embodiment.

FIG. 4 is a conceptual diagram for describing an example of a relationship between a local coordinate system and a world coordinate system set in the gait measurement device according to the first example embodiment.

FIG. 5 is a conceptual diagram for describing human body planes used in the description regarding the gait measurement device according to the first example embodiment.

FIG. 6 is a conceptual diagram for describing a gait cycle used in the description regarding the gait measurement device according to the first example embodiment.

FIG. 7 is a conceptual diagram for describing gait parameters used in the description regarding the gait measurement device according to the first example embodiment.

FIG. 8 is a graph for describing an example of time-series data of sensor data measured by the gait measurement device according to the first example embodiment.

FIG. 9 is a diagram for describing an example of normalization of gait waveform data extracted from the time-series data of the sensor data measured by the gait measurement device according to the first example embodiment.

FIG. 10 is a conceptual diagram for describing an example of a gait phase cluster from which a feature amount data generation unit of the gait measurement device according to the first example embodiment extracts a feature amount.

FIG. 11 is a block diagram illustrating an example of a configuration of a fall probability estimation device included in the fall probability estimation system according to the first example embodiment.

FIG. 12 is a conceptual diagram for describing items related to the fall probability to be estimated by the fall probability estimation system according to the first example embodiment.

FIG. 13 is a table summarizing feature amounts regarding a total muscular strength (grip strength) of a whole body related to the fall probability to be estimated by the fall probability estimation system according to the first example embodiment.

FIG. 14 is a table summarizing feature amounts regarding a dynamic balance related to the fall probability to be estimated by the fall probability estimation system according to the first example embodiment.

FIG. 15 is a table summarizing feature amounts regarding a lower-limb muscular strength related to the fall probability to be estimated by the fall probability estimation system according to the first example embodiment.

FIG. 16 is a table summarizing feature amounts regarding a mobility related to the fall probability to be estimated by the fall probability estimation system according to the first example embodiment.

FIG. 17 is a table summarizing feature amounts regarding a static balance related to the fall probability to be estimated by the fall probability estimation system according to the first example embodiment.

FIG. 18 is a table summarizing feature amounts regarding fall probability to be estimated by the fall probability estimation system according to the first example embodiment.

FIG. 19 is a conceptual diagram illustrating an example of estimation of a fall probability score (fall probability index) by the fall probability estimation device included in the fall probability estimation system according to the first example embodiment.

FIG. 20 is a conceptual diagram illustrating an example of estimation of a fall probability score (fall probability index) by the fall probability estimation device included in the fall probability estimation system according to the first example embodiment.

FIG. 21 is a flowchart for describing an example of an operation of the gait measurement device included in the fall probability estimation system according to the first example embodiment.

FIG. 22 is a flowchart for describing an example of an operation of the fall probability estimation device included in the fall probability estimation system according to the first example embodiment.

FIG. 23 is a flowchart for describing an example of an operation of the gait measurement device included in the fall probability estimation system according to the first example embodiment.

FIG. 24 is a flowchart for describing an example of an operation of the fall probability estimation device included in the fall probability estimation system according to the first example embodiment.

FIG. 25 is a conceptual diagram for describing an application example of the fall probability estimation system according to the first example embodiment.

FIG. 26 is a conceptual diagram for describing an application example of the fall probability estimation system according to the first example embodiment.

FIG. 27 is a block diagram illustrating an example of a configuration of a machine learning system according to a second example embodiment.

FIG. 28 is a block diagram illustrating an example of a configuration of a machine learning device included in the machine learning system according to the second example embodiment.

FIG. 29 is a conceptual diagram for describing an example of machine learning by the machine learning device included in the machine learning system according to the second example embodiment.

FIG. 30 is a conceptual diagram for describing another example of machine learning by the machine learning device included in the machine learning system according to the second example embodiment.

FIG. 31 is a block diagram illustrating an example of a configuration of a fall probability estimation device according to a third example embodiment.

FIG. 32 is a block diagram illustrating an example of a hardware configuration that executes control and processing according to each example embodiment.

### EXAMPLE EMBODIMENT

Hereinafter, example embodiments of the present invention will be described with reference to the drawings. However, the example embodiments described below have technically preferable limitations for carrying out the present invention, but do not limit the scope of the invention to those described below. In all the drawings used in the description of the example embodiments below, the same reference numerals are given to the same parts unless there is a particular reason. In the example embodiments described below, repeated description of similar configurations and operations may be omitted.

### First Example Embodiment

First, a fall probability estimation system according to a first example embodiment will be described with reference to the drawings. The fall probability estimation system according to the present example embodiment measures sensor data regarding a motion of a foot according to gait of a user. The fall probability estimation system of the present example embodiment estimates the fall probability of the user using the measured sensor data.

In the present example embodiment, an example of estimating the fall probability according to the relevance between a feature (also referred to as gait) included in a gait pattern and the fall risk will be described. The fall probability, which is one of the indices of the fall risk, can be evaluated based on the variability of gait parameters. In the present example embodiment, the fall probability is estimated using five related items (also referred to as the five items) related to the gait. The five items relate to the total muscular strength (grip strength) of the whole body, the dynamic balance, the lower-limb muscular strength, the mobility, and the static balance. These five items have a correlation with the fall probability. The five items are considered to be related to each other to some extent but basically independent of each other. In the present example embodiment, an example is given in which the fall probability is estimated based on all of the five items, but the fall probability can be estimated based on at least one of the five items.

### (Configuration)

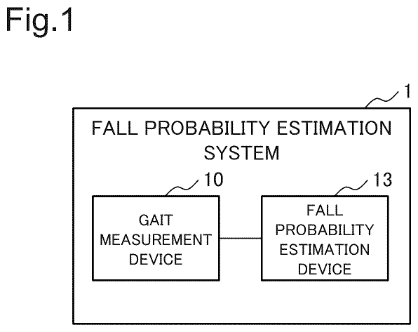

FIG. 1 is a block diagram illustrating an example of a configuration of a fall probability estimation system 1 according to the present example embodiment. The fall probability estimation system 1 includes a gait measurement device 10 and a fall probability estimation device 13. In the present example embodiment, an example in which the gait measurement device 10 and the fall probability estimation device 13 are configured as separate hardware will be described. For example, the gait measurement device 10 is installed in footwear or the like of a subject (user) who is a target for estimation of the fall probability. For example, the function of the fall probability estimation device 13 is installed in a mobile terminal carried by a subject (user). Hereinafter, configurations of the gait measurement device 10 and the fall probability estimation device 13 will be individually described.

### [Gait Measurement Device]

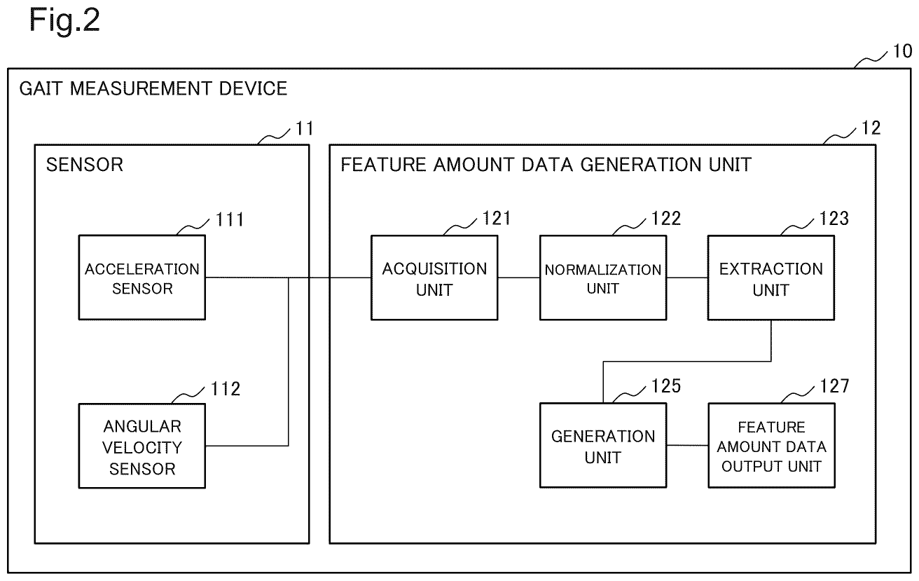

FIG. 2 is a block diagram illustrating an example of a configuration of the gait measurement device 10. The gait measurement device 10 includes a sensor 11 and a feature amount data generation unit 12. In the present example embodiment, an example in which the sensor 11 and the feature amount data generation unit 12 are integrated will be described. The sensor 11 and the feature amount data generation unit 12 may be provided as separate devices.

As illustrated in FIG. 2, the sensor 11 includes an acceleration sensor 111 and an angular velocity sensor 112. FIG. 2 illustrates an example in which the acceleration sensor 111 and the angular velocity sensor 112 are included in the sensor 11. The sensor 11 may include a sensor other than the acceleration sensor 111 and the angular velocity sensor 112. Sensors other than the acceleration sensor 111 and the angular velocity sensor 112 that can be included in the sensor 11 will not be described.

The acceleration sensor 111 is a sensor that measures accelerations (also referred to as spatial accelerations) in three axial directions. The acceleration sensor 111 measures accelerations (also referred to as spatial accelerations) as a physical quantity regarding the motion of the foot. The acceleration sensor 111 outputs the measured acceleration to the feature amount data generation unit 12. For example, a sensor of a piezoelectric type, a piezoresistive type, a capacitance type, or the like can be used as the acceleration sensor 111. The measurement method of the sensor used as the acceleration sensor 111 is not limited as long as the sensor can measure the acceleration.

The angular velocity sensor 112 is a sensor that measures angular velocities (also referred to as spatial angular velocities) around three axes. The angular velocity sensor 112 measures angular velocities (also referred to as spatial angular velocities) as a physical quantity regarding the motion of the foot. The angular velocity sensor 112 outputs the measured angular velocity to the feature amount data generation unit 12. For example, a sensor of a vibration type, a capacitance type, or the like can be used as the angular velocity sensor 112. The measurement method of the sensor used as the angular velocity sensor 112 is not limited as long as the sensor can measure the angular velocity.

The sensor 11 is achieved by, for example, an inertial measurement device that measures the acceleration and the angular velocity. An inertial measurement unit (IMU) is an example of the inertial measurement device. The IMU includes the acceleration sensor 111 that measures the accelerations in the three axial directions and the angular velocity sensor 112 that measures the angular velocities around the three axes. The sensor 11 may be achieved by an inertial measurement device such as a vertical gyro (VG) or an attitude heading (AHRS). The sensor 11 may be achieved by a global positioning system/inertial navigation system (GPS/INS). The sensor 11 may be achieved by a device other than the inertial measurement device as long as it can measure a physical quantity regarding the motion of the foot.

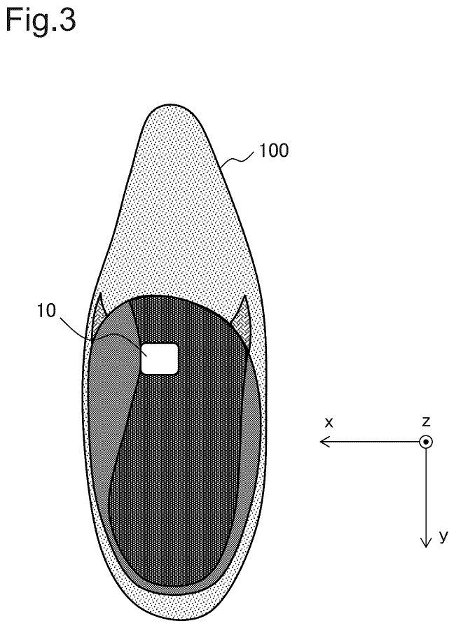

FIG. 3 is a conceptual diagram illustrating an example in which the gait measurement device 10 is arranged in a shoe 100 of a right foot. In the example of FIG. 3, the gait measurement device 10 is installed at a position corresponding to the back side of the arch of the foot. For example, the gait measurement device 10 is arranged on an insole inserted into the shoe 100. For example, the gait measurement device 10 may be arranged on the bottom surface of the shoe 100. For example, the gait measurement device 10 may be embedded in the main body of the shoe 100. The gait measurement device 10 may be detachable from the shoe 100 or may not be detachable from the shoe 100. The gait measurement device 10 may be installed at a position other than the back side of the arch of the foot as long as sensor data regarding the motion of the foot can be measured. The gait measurement device 10 may be installed on a sock worn by the user or a decorative article such as an anklet worn by the user. The gait measurement device 10 may be directly attached to the foot or may be embedded in the foot. FIG. 3 illustrates an example in which the gait measurement device 10 is installed in the shoe 100 of the right foot. The gait measurement device 10 may be installed in the shoes 100 of both feet.

In the example of FIG. 3, a local coordinate system including an x axis in a left-right direction, a y axis in a front-rear direction, and a z axis in an up-down direction is set with reference to the gait measurement device 10 (sensor 11). In the x-axis, the left side is positive, in the y-axis, the rear side is positive, and in the z-axis, the upper side is positive. The direction of the axis set in the sensor 11 may be the same for the left and right feet, or may be different for the left and right feet. For example, in a case where the sensors 11 produced with the same specs are arranged in the left and right shoes 100, the up-down directions (directions in the Z-axis direction) of the sensors 11 arranged in the left and right shoes 100 are the same. In this case, the three axes of the local coordinate system set in the sensor data derived from the left foot and the three axes of the local coordinate system set in the sensor data derived from the right foot are the same for the left and right.

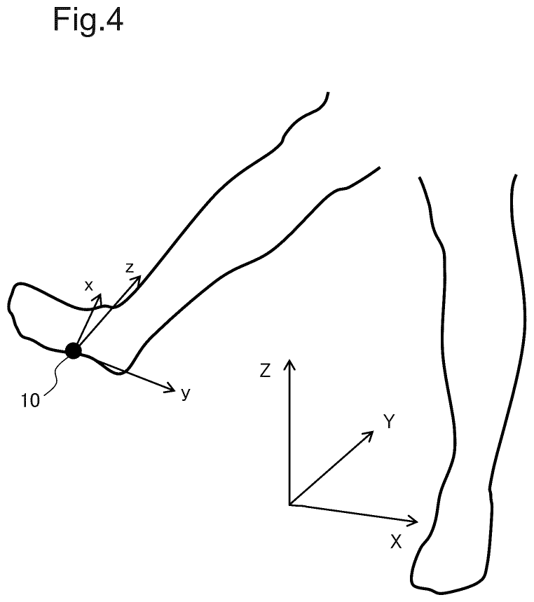

FIG. 4 is a conceptual diagram for describing a local coordinate system (x-axis, y-axis, z-axis) set in the gait measurement device 10 (sensor 11) installed on the back side of the arch of the foot and a world coordinate system (X-axis, Y-axis, Z-axis) set with respect to the ground. In the world coordinate system (X-axis, Y-axis, Z-axis), in a state where the user facing the traveling direction is upright, the lateral direction of the user is set to the X-axis direction (the leftward direction is positive), the direction of the back surface of the user is set to the Y-axis direction (the rearward direction is positive), and the gravity direction is set to the Z-axis direction (the vertically upward direction is positive). The example of FIG. 4 conceptually illustrates the relationship between the local coordinate system (x-axis, y-axis, z-axis) and the world coordinate system (X-axis, Y-axis, Z-axis), and does not accurately illustrate the relationship between the local coordinate system and the world coordinate system that varies depending on the gait of the user.

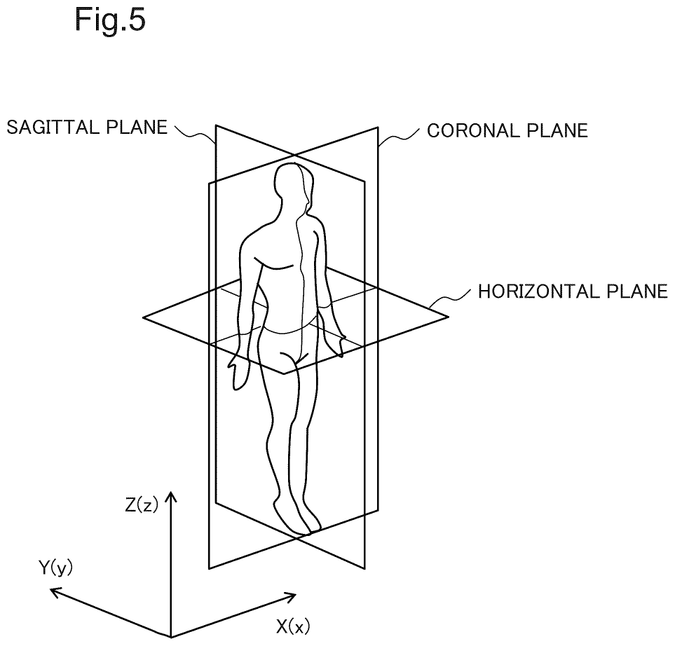

FIG. 5 is a conceptual diagram for describing planes (also referred to as human body planes) set for a human body. In the present example embodiment, a sagittal plane dividing the body into left and right, a coronal plane dividing the body into front and rear, and a horizontal plane dividing the body horizontally are defined. As illustrated in FIG. 5, the world coordinate system and the local coordinate system coincide with each other in the upright state with the center line of the foot oriented in the traveling direction. In the present example embodiment, rotation in the sagittal plane with the x-axis as the rotation axis is defined as roll, rotation in the coronal plane with the y-axis as the rotation axis is defined as pitch, and rotation in the horizontal plane with the z-axis as the rotation axis is defined as yaw. A rotation angle in the sagittal plane with the x-axis as the rotation axis is defined as a roll angle, a rotation angle in the coronal plane with the y-axis as the rotation axis is defined as a pitch angle, and a rotation angle in the horizontal plane with the z-axis as the rotation axis is defined as a yaw angle.

As illustrated in FIG. 2, the feature amount data generation unit 12 (also referred to as a feature amount data generation device) includes an acquisition unit 121, a normalization unit 122, an extraction unit 123, a generation unit 125, and a feature amount data output unit 127. For example, the feature amount data generation unit 12 is achieved by a microcomputer or a microcontroller that performs overall control and data processing of the gait measurement device 10. For example, the feature amount data generation unit 12 includes a central processing unit (CPU), random access memory (RAM), read only memory (ROM), flash memory, and the like. The feature amount data generation unit 12 controls the acceleration sensor 111 and the angular velocity sensor 112 to measure the angular velocity and the acceleration. For example, the feature amount data generation unit 12 may be mounted on a mobile terminal (not illustrated) carried by a subject (user).

The acquisition unit 121 acquires the accelerations in the three axial directions from the acceleration sensor 111. The acquisition unit 121 acquires the angular velocities around the three axes from the angular velocity sensor 112. For example, the acquisition unit 121 performs analog-to-digital conversion (AD conversion) on the acquired physical quantities (analog data) such as the angular velocity and the acceleration. The physical quantities (analog data) measured by the acceleration sensor 111 and the angular velocity sensor 112 may be converted into digital data in each of the acceleration sensor 111 and the angular velocity sensor 112. The acquisition unit 121 outputs the converted digital data (also referred to as sensor data) to the normalization unit 122. The acquisition unit 121 may be configured to cause a storage unit, which is not illustrated, to store the sensor data. The sensor data includes at least acceleration data converted into digital data and angular velocity data converted into digital data. The acceleration data includes acceleration vectors in the three axial directions. The angular velocity data includes angular velocity vectors around the three axes. The acceleration data and the angular velocity data are associated with acquisition times of the data. The acquisition unit 121 may add correction such as a mounting error, temperature correction, and linearity correction to the acceleration data and the angular velocity data.

The normalization unit 122 acquires the sensor data from the acquisition unit 121. The normalization unit 122 extracts time-series data (also referred to as gait waveform data) for one gait cycle from the time-series data of the accelerations in the three axial directions and the angular velocities around the three axes included in the sensor data. The normalization unit 122 normalizes (also referred to as first-normalizes) the time of the extracted gait waveform data for one gait cycle to a gait cycle of 0 to 100% (percent). Timing such as 1% or 10% included in the gait cycle of 0 to 100% is also referred to as a gait phase. The normalization unit 122 normalizes (also referred to as second-normalizes) the first-normalized gait waveform data for one gait cycle in such a way that the stance phase becomes 60% and the swing phase becomes 40%. The stance phase is a period in which at least a part of the back side of the foot is in contact with the ground. The swing phase is a period in which the back side of the foot is away from the ground. When the gait waveform data is subjected to the second normalization, it is possible to suppress a deviation of the gait phase from which the feature amount is extracted from fluctuating due to the effect of disturbance.

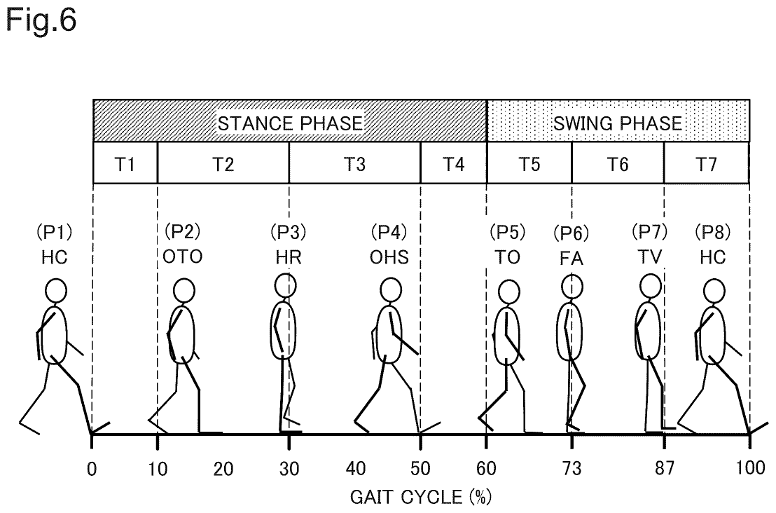

FIG. 6 is a conceptual diagram for describing one gait cycle based on the right foot. One gait cycle based on the left foot is similar to that of the right foot. The horizontal axis in FIG. 6 is one gait cycle of the right foot with a time point at which the heel of the right foot lands on the ground as a starting point and a time point at which the heel of the right foot next lands on the ground as an ending point. The horizontal axis in FIG. 6 is first-normalized with one gait cycle as 100%. The horizontal axis in FIG. 6 is second-normalized in such a way that the stance phase is 60% and the swing phase is 40%. The one gait cycle of one foot is roughly divided into the stance phase in which at least a part of the back side of the foot is in contact with the ground and the swing phase in which the back side of the foot is away from the ground. The stance phase is further subdivided into a load response period T1, a mid-stance period T2, a terminal stance period T3, and a pre-swing period T4. The swing phase is further subdivided into an initial swing period T5, a mid-swing period T6, and a terminal swing period T7. FIG. 6 is an example, and does not limit the periods constituting the one gait cycle, the names of these periods, and the like.

As illustrated in FIG. 6, in gait, a plurality of events (also referred to as gait events) occur. P1 indicates an event in which the heel of the right foot touches the ground (heel contact (HC)). P2 indicates an event in which the toe of the left foot is away from the ground with the sole of the right foot in contact with the ground (opposite toe off (OTO)). P3 indicates an event in which the heel of the right foot rises with the sole of the right foot in contact with the ground (heel rise (HR)). P4 is an event in which the heel of the left foot touches the ground (opposite heal strike (OHS)). P5 indicates an event in which the toe of the right foot is away from the ground with the sole of the left foot in contact with the ground (toe off (TO)). P6 indicates an event in which the left foot and the right foot cross with the sole of the left foot in contact with the ground (foot adjacent (FA). P7 indicates an event in which the tibia of the right foot is approximately perpendicular to the ground with the sole of the left foot in contact with the ground (tibia vertical (TV)). P8 indicates an event in which the heel of the right foot touches the ground (heel contact (HC)). P8 corresponds to the ending point of the gait cycle starting from P1 and corresponds to the starting point of the next gait cycle. FIG. 6 is an example, and does not limit the events that occur during gait or the names of these events.

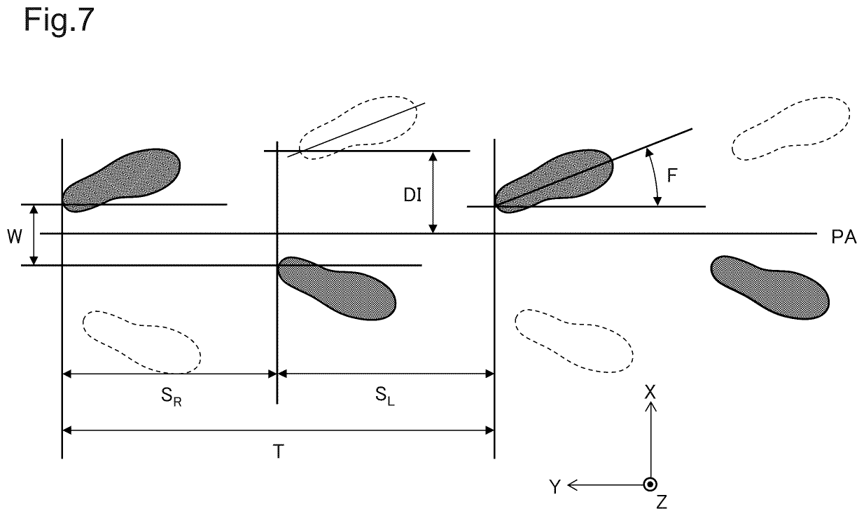

FIG. 7 is a conceptual diagram for describing an example of gait parameters. FIG. 7 illustrates a right foot step length S_{R}, a left foot step length S_{L}, a stride length T, a step width W, a foot angle F, and a circumduction amount D1. FIG. 7 illustrates a traveling axis PA that is parallel to the axis (Y axis) in the traveling direction and corresponds to a trajectory connecting the middle of the left and right feet. The right foot step length S_{R} is a difference in the Y coordinate between the heel of the right foot and the heel of the left foot when the state in which the sole of the left foot is in contact with the ground transitions to the state in which the heel of the right foot swung out in the traveling direction lands. The left foot step length S_{L} is a difference in the Y coordinate between the heel of the left foot and the heel of the right foot when the state in which the sole of the right foot is in contact with the ground transitions to the state in which the heel of the left foot swung out in the traveling direction lands. The stride length T is the sum of the right foot step length S_{R} and the left foot step length S_{L}. The step width W is an interval between the right foot and the left foot. In FIG. 7, the step width W is a difference between the center line (X-coordinate) of the heel of the right foot in the grounded state and the center line (X-coordinate) of the heel of the left foot in the grounded state. The foot angle F is an angle formed by the center line of the foot and the traveling direction (Y-axis) in a state where the back surface of the foot is in contact with the ground. In the present example embodiment, the foot angle in a state where the foot is in contact with the ground is evaluated in the stance phase. The circumduction amount D1 is a distance between the traveling axis PA and the foot at a timing when the central axis of the foot is farthest from the traveling axis PA in the swing phase. In the present example embodiment, since the length of the lower limb affects the circumduction amount D1, the circumduction amount D1 is normalized by the height.

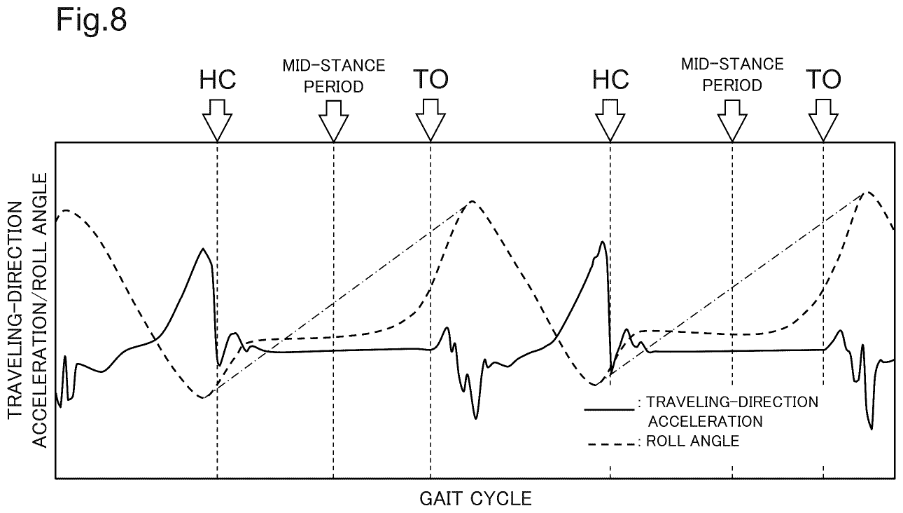

FIG. 8 is a diagram for describing an example of detecting the heel contact HC and the toe off TO from the time-series data (solid line) of the traveling-direction acceleration (Y-direction acceleration). The timing of the heel contact HC is the timing of the minimum peak immediately after the maximum peak appearing in the time-series data of the traveling-direction acceleration (Y-direction acceleration). The maximum peak serving as a mark of the timing of the heel contact HC corresponds to the largest peak of the gait waveform data for one gait cycle. A section between the consecutive pieces of heel contact HC is one gait cycle. The timing of the toe off TO is the timing of rising of the maximum peak appearing after the period of the stance phase in which the fluctuation does not appear in the time-series data of the traveling-direction acceleration (Y-direction acceleration). FIG. 8 also illustrates time-series data (broken line) of the roll angle (angular velocity around the X axis). The timing at the midpoint between the timing at which the roll angle is minimum and the timing at which the roll angle is maximum corresponds to the mid-stance period. For example, parameters (also referred to as gait parameters) such as gait speed, stride, circumduction, medial rotation/external rotation, and plantarflexion/dorsiflexion can be obtained with reference to the mid-stance period.

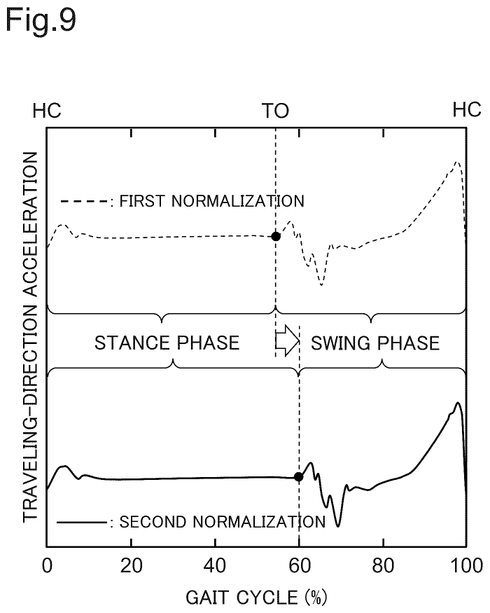

FIG. 9 is a diagram for describing an example of the gait waveform data normalized by the normalization unit 122. The normalization unit 122 detects the heel contact HC and the toe off TO from the time-series data of the traveling-direction acceleration (Y-direction acceleration). The normalization unit 122 extracts the section between the consecutive pieces of heel contact HC as gait waveform data for one gait cycle. The normalization unit 122 converts the horizontal axis (time axis) of the gait waveform data for one gait cycle into a gait cycle of 0 to 100% by the first normalization. In FIG. 9, the gait waveform data after the first normalization is indicated by the broken line. In the gait waveform data (broken line) after the first normalization, the timing of the toe off TO deviates from 60%.

In the example of FIG. 9, the normalization unit 122 normalizes a section from the heel contact HC in which the gait phase is 0% to the toe off TO subsequent to the heel contact HC to 0 to 60%. The normalization unit 122 normalizes a section from the toe off TO to the heel contact HC in which the gait phase is 100% subsequent to the toe off TO to 60 to 100%. As a result, the gait waveform data for one gait cycle is normalized to a section (stance phase) in which the gait cycle is 0 to 60% and a section (swing phase) in which the gait cycle is 60 to 100%. In FIG. 9, the gait waveform data after the second normalization is indicated by the solid line. In the gait waveform data (solid line) after the second normalization, the timing of the toe off TO coincides with 60%.

FIGS. 8 to 9 illustrate examples in which the gait waveform data for one gait cycle is extracted/normalized based on the traveling-direction acceleration (Y-direction acceleration). With respect to acceleration other than the traveling-direction acceleration (Y-direction acceleration)/angular velocity, the normalization unit 122 extracts/normalizes the gait waveform data for one gait cycle in accordance with the gait cycle of the traveling-direction acceleration (Y-direction acceleration). The normalization unit 122 may generate time-series data of the angles around the three axes by integrating the time-series data of the angular velocities around the three axes. In that case, also regarding the angles around the three axes, the normalization unit 122 extracts/normalizes the gait waveform data for one gait cycle in accordance with the gait cycle of the traveling-direction acceleration (Y-direction acceleration).

The normalization unit 122 may extract/normalize the gait waveform data for one gait cycle based on acceleration other than the traveling-direction acceleration (Y-direction acceleration)/angular velocity (the drawings are omitted). For example, the normalization unit 122 may detect the heel contact HC and the toe off TO from the time-series data of the perpendicular-direction acceleration (Z-direction acceleration). The timing of the heel contact HC is the timing of the steep minimum peak appearing in the time-series data of the perpendicular-direction acceleration (Z-direction acceleration). At the timing of the steep minimum peak, the value of the perpendicular-direction acceleration (Z-direction acceleration) becomes substantially zero. The minimum peak serving as a mark of the timing of the heel contact HC corresponds to the smallest peak of the gait waveform data for one gait cycle. A section between the consecutive pieces of heel contact HC is one gait cycle. The timing of the toe off TO is a timing of an inflection point in the middle of a gradual increase of the time-series data of the perpendicular-direction acceleration (Z-direction acceleration) after passing through a section with a small fluctuation after the maximum peak immediately after the heel contact HC. The normalization unit 122 may extract/normalize the gait waveform data for one gait cycle based on both the traveling-direction acceleration (Y-direction acceleration) and the perpendicular-direction acceleration (Z-direction acceleration). The normalization unit 122 may extract/normalize the gait waveform data for one gait cycle based on the acceleration other than the traveling-direction acceleration (Y-direction acceleration) and the perpendicular-direction acceleration (Z-direction acceleration), the angular velocity, the angle, and the like.

The extraction unit 123 acquires the gait waveform data for one gait cycle normalized by the normalization unit 122. The extraction unit 123 extracts a feature amount used for estimation of the fall probability from the gait waveform data for one gait cycle. The extraction unit 123 extracts a feature amount for each gait phase cluster from a gait phase cluster obtained by integrating temporally continuous gait phases based on a preset condition. The gait phase cluster includes at least one gait phase. The gait phase cluster also includes a single gait phase. The gait waveform data and the gait phase from which the feature amount used for estimating the fall probability is extracted will be described below.

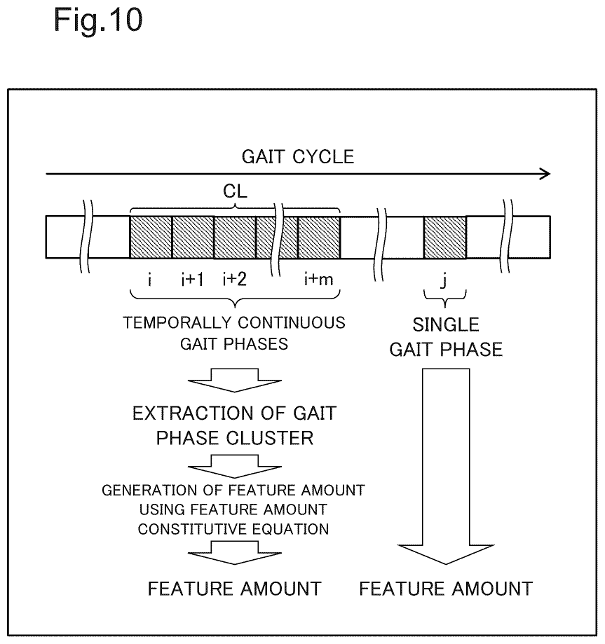

FIG. 10 is a conceptual diagram for describing extraction of a feature amount for estimating fall probability from gait waveform data for one gait cycle. For example, the extraction unit 123 extracts temporally continuous gait phases i to i+m as a gait phase cluster CL (i and m are natural numbers). The gait phase cluster CL includes m gait phases (components). That is, the number of gait phases (components) (also referred to as the number of components) constituting the gait phase cluster CL is m. FIG. 10 illustrates an example in which the gait phase has an integer value, but the gait phase may be subdivided into decimal places. When the gait phase is subdivided into decimal places, the number of components of the gait phase cluster CL is a number related to the number of pieces of data in the section of the gait phase cluster. The extraction unit 123 extracts a feature amount from each of the gait phases i to i+m. In a case where the gait phase cluster CL includes a single gait phase j, the extraction unit 123 extracts a feature amount from the single gait phase j (j is a natural number).

The generation unit 125 applies a feature amount constitutive equation to the feature amount (first feature amount) extracted from each of the gait phases constituting the gait phase cluster to generate the feature amount (second feature amount) of the gait phase cluster. The feature amount constitutive equation is a preset calculation expression for generating the feature amount of the gait phase cluster. For example, the feature amount constitutive equation is a calculation expression related to four arithmetic operations. For example, the second feature amount calculated using the feature amount constitutive equation is an integral average value, an arithmetic average value, a slope, a variation, or the like of the first feature amount in each gait phase included in the gait phase cluster. For example, the generation unit 125 applies the calculation expression for calculating the slope and variation of the first feature amount extracted from each of the gait phases constituting the gait phase cluster as the feature amount constitutive equation. For example, in a case where the gait phase cluster includes a single gait phase, it is not possible to calculate the slope or variation, and thus, it is sufficient to use the feature amount constitutive equation for calculating an integral average value, an arithmetic average value, or the like.

The feature amount data output unit 127 outputs the feature amount data for each gait phase cluster generated by the generation unit 125. The feature amount data output unit 127 outputs the generated feature amount data of the gait phase cluster to the fall probability estimation device 13 using the feature amount data.

### [Fall Probability Estimation Device]

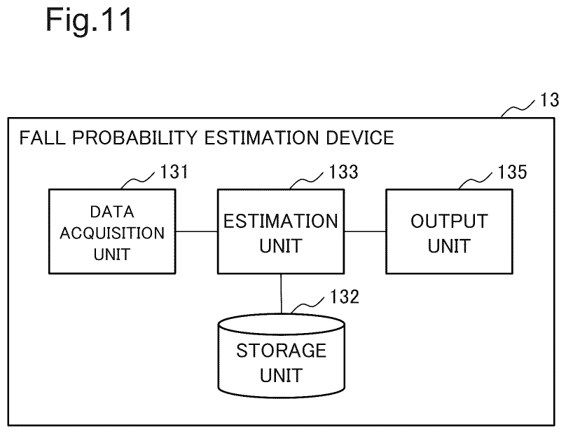

FIG. 11 is a block diagram illustrating an example of a configuration of the fall probability estimation device 13. The fall probability estimation device 13 includes a data acquisition unit 131, a storage unit 132, an estimation unit 133, and an output unit 135.

The data acquisition unit 131 acquires the feature amount data from the gait measurement device 10. The data acquisition unit 131 outputs the received feature amount data to the estimation unit 133. The data acquisition unit 131 may receive the feature amount data from the gait measurement device 10 via a wire such as a cable, or may receive the feature amount data from the gait measurement device 10 via wireless communication. For example, the data acquisition unit 131 is configured to receive the feature amount data from the gait measurement device 10 via a wireless communication function (not illustrated) conforming to a standard such as Bluetooth (registered trademark) or Wi-Fi (registered trademark). The communication function of the data acquisition unit 131 may conform to a standard other than Bluetooth (registered trademark) or Wi-Fi (registered trademark).

The storage unit 132 stores an estimation model for estimating the fall probability using the feature amount data extracted from the gait waveform data. The storage unit 132 stores an estimation model for estimating the fall probability machine-learned regarding a plurality of subjects. For example, the storage unit 132 stores an estimation model that outputs a fall probability index (also referred to as a fall probability score) according to the input of the feature amount data extracted from the gait waveform data.

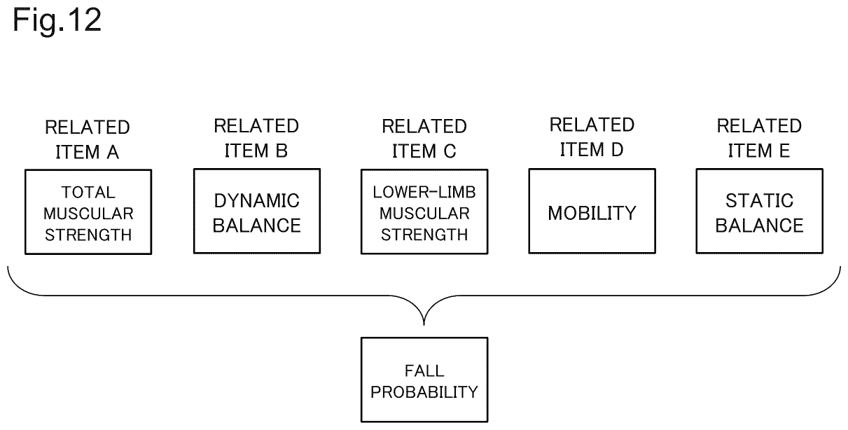

FIG. 12 is a conceptual diagram for describing five related items (also referred to as five items) related to the fall probability. The five items of the total muscular strength (grip strength) of the whole body, the dynamic balance, the lower-limb muscular strength, the mobility, and the static balance relate to the fall probability. Details of the five items of the total muscular strength (grip strength) of the whole body, the dynamic balance, the lower-limb muscular strength, the mobility, and the static balance related to the fall probability will be described below.

For example, the storage unit 132 stores an estimation model (also referred to as first estimation model) that outputs a fall probability index (fall probability score) according to the input of the feature amount data common to the estimation of the five items. For example, the storage unit 132 stores an estimation model (also referred to as a pre-estimation model) that outputs the score of each of the five items according to the input of the feature amount data used to estimate the score of each of the five items. For example, the storage unit 132 stores an estimation model (also referred to as second estimation model) that outputs a fall probability index (fall probability score) according to the input of the scores of the five items.

It is sufficient if the estimation model is stored in the storage unit 132 at the time of shipment of a product from a factory, calibration before the user uses the fall probability estimation system 1, or the like. For example, an estimation model stored in a storage device such as an external server may be used. In that case, it is sufficient if the estimation model is configured to be used via an interface (not illustrated) connected to the storage device.

The estimation unit 133 acquires the feature amount data from the data acquisition unit 131. The estimation unit 133 executes estimation of the fall probability using the acquired feature amount data. The estimation unit 133 inputs the feature amount data to the estimation model stored in the storage unit 132. The estimation unit 133 outputs an estimation result according to the fall probability output from the estimation model. In a case where an estimation model stored in an external storage device constructed in a cloud, a server, or the like is used, the estimation unit 133 is configured to use the estimation model via an interface (not illustrated) connected to the storage device.

The output unit 135 outputs an estimation result of the fall probability by the estimation unit 133. For example, the output unit 135 displays the estimation result of the fall probability on the screen of the mobile terminal of the subject (user). For example, the output unit 135 outputs the estimation result to an external system or the like that uses the estimation result. The use of the fall probability output from the fall probability estimation device 13 is not particularly limited.

For example, the fall probability estimation device 13 is connected to an external system or the like constructed in a cloud or a server via a mobile terminal (not illustrated) carried by a subject (user). The mobile terminal (not illustrated) is a portable communication device. For example, the mobile terminal is a portable communication device having a communication function, such as a smartphone, a smart watch, or a mobile phone. For example, the fall probability estimation device 13 is connected to the mobile terminal via a wire such as a cable. For example, the fall probability estimation device 13 is connected to the mobile terminal via wireless communication. For example, the fall probability estimation device 13 is connected to the mobile terminal via a wireless communication function (not illustrated) conforming to a standard such as Bluetooth (registered trademark) or Wi-Fi (registered trademark). The communication function of the fall probability estimation device 13 may conform to a standard other than Bluetooth (registered trademark) or Wi-Fi (registered trademark). The estimation result of the fall probability may be used by an application installed in the mobile terminal. In that case, the mobile terminal executes processing using the estimation result by application software or the like installed in the mobile terminal.

Next, each of the five items of the total muscular strength (grip strength) of the whole body, the dynamic balance, the lower-limb muscular strength, the mobility, and the static balance related to the fall probability illustrated in FIG. 12 will be individually described below. After each of the five items is described, the feature amount used for estimating the fall probability will be described. The fall probability is estimated using the feature amount common to the five items.

### <Related Item A>

Related item A relates to the total muscular strength of the whole body. There is a correlation between the total muscular strength and the grip strength. The grip strength is also correlated with the knee extension strength. One of indices of the total muscular strength related to the related item A is grip strength. For example, an estimation value of the grip strength is an index of the total muscular strength. For example, a score according to the estimation value of the grip strength (also referred to as a total muscular strength score) is an index of the total muscular strength. The total muscular strength score is a value obtained by scoring the grip strength, which is an index of the total muscular strength, on a preset basis. The grip strength is affected by attributes such as sex, age, and height. Therefore, the total muscular strength score may be scored with reference to each attribute. In particular, the grip strength is affected by sex. Therefore, the total muscular strength score may be scored based on criteria different depending on the sex. The index of the total muscular strength is not limited to the grip strength as long as the total muscular strength can be scored.

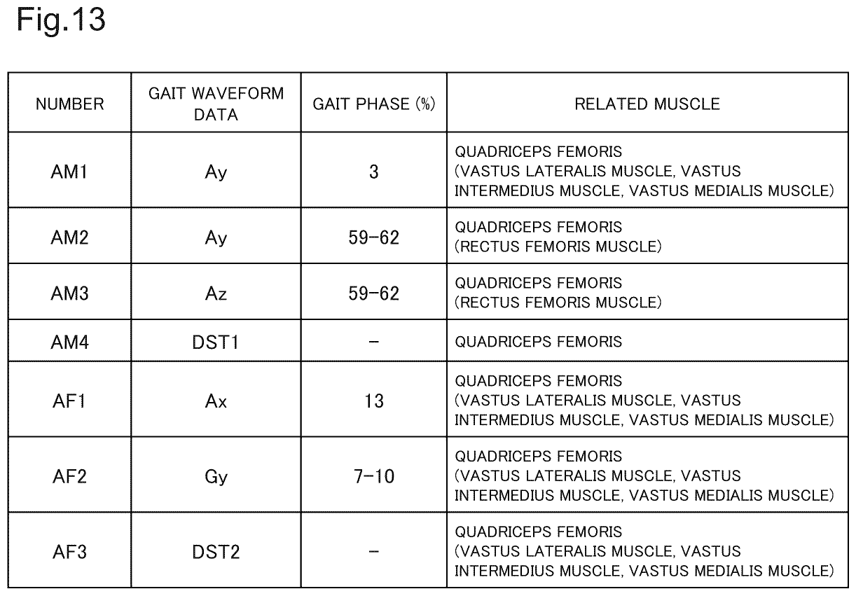

FIG. 13 is a correspondence table summarizing feature amounts used to estimate the total muscular strength (grip strength) of the whole body. The correspondence table in FIG. 13 associates the number of the feature amount, the gait waveform data from which the feature amount is extracted, the gait phase (%) from which the gait phase cluster is extracted, and the related muscle. The gait phase from which the feature amount used to estimate the grip strength is extracted differs depending on the sex. In the case of a male, there is a correlation between the activity of quadriceps femoris and the grip strength. Therefore, feature amounts AM1 to AM4 extracted from the gait phase in which the feature of the activity of the quadriceps femoris appears are used to estimate the grip strength of the male. In the case of a female, there is a correlation between the activities of the vastus lateralis muscle, the vastus intermedius muscle, and the vastus medialis muscle of the quadriceps femoris and the grip strength. Therefore, feature amounts AF1 to AF3 extracted from the gait phase in which the feature of the activities of the vastus lateralis muscle, the vastus intermedius muscle, and the vastus medialis muscle appears are used to estimate the grip strength of the female.

The feature amount AM1 is extracted from a section of gait phase 3% of gait waveform data Ay related to the time-series data of the traveling-direction acceleration (Y-direction acceleration). The gait phase 3% is included in the load response period T1. The feature amount AM1 mainly includes features regarding the motion of the vastus lateralis muscle, the vastus intermedius muscle, and the vastus medialis muscle of the quadriceps femoris.

The feature amount AM2 is extracted from a section of gait phase 59 to 62% of gait waveform data Ay related to the time-series data of the traveling-direction acceleration (Y-direction acceleration). The gait phase 59 to 62% is included in the pre-swing period T4. The feature amount AM2 mainly includes features regarding the motion of the rectus femoris muscle of the quadriceps femoris.

The feature amount AM3 is extracted from a section of gait phase 59 to 62% of gait waveform data Az related to the time-series data of the perpendicular-direction acceleration (Z-direction acceleration). The gait phase 59 to 62% is included in the pre-swing period T4. The feature amount AM3 mainly includes features regarding the motion of the rectus femoris muscle of the quadriceps femoris.

The feature amount AM4 is a ratio of a period from the heel contact to the opposite toe off within the period in which both feet simultaneously touch the ground (DST1) (double support time (DST)). DST1 is a ratio of a period from the heel contact to the opposite toe off in one gait cycle. The feature amount AM4 mainly includes a feature caused by the quadriceps femoris.

The feature amount AF1 is extracted from a section of gait phase 13% of gait waveform data Ax related to the time-series data of the lateral-direction acceleration (X-direction acceleration). The gait phase 13% is included in the mid-stance period T2. The feature amount AF1 mainly includes features regarding the motion of the vastus lateralis muscle, the vastus intermedius muscle, and the vastus medialis muscle of the quadriceps femoris.

The feature amount AF2 is extracted from a section of gait phase 7 to 10% of gait waveform data Gy related to the time-series data of the angular velocity (pitch angular velocity) in the coronal plane (around the Y axis). The gait phase 7 to 10% is included in the load response period T1. The feature amount AF2 mainly includes features regarding the motion of the vastus lateralis muscle, the vastus intermedius muscle, and the vastus medialis muscle.

The feature amount AF3 is a ratio of a period from the opposite heal strike to the toe off within the period in which both feet simultaneously touch the ground (DST2) (double support time (DST)). DST2 is a ratio of a period from the opposite heal strike to the toe off in one gait cycle. The sum of DST1 and DST2 corresponds to a period in which both feet simultaneously touch the ground in one gait cycle. The feature amount AF3 mainly includes features regarding the motion of the vastus lateralis muscle, the vastus intermedius muscle, and the vastus medialis muscle.

### <Related Item B>

The related item B relates to the dynamic balance. The dynamic balance can be evaluated by a result of functional reach test (FRT). In the present example embodiment, the FRT result is evaluated by the distance between the fingertips (also referred to as functional reach distance) in a state where the upper limb is moved forward as much as possible from an upright state with both hands raised 90 degrees with respect to the horizontal plane. The functional reach distance (hereinafter, referred to as an FR distance) is a result value of the FRT. The larger the FR distance, the higher the FRT result. The related item B may be evaluated other than the FRT performed with both hands. For example, the related item B may be evaluated in terms of results regarding one-handed FRT or other variations of the FRT.

The index of the dynamic balance regarding the related item B is the FR distance. For example, the estimation value of the FR distance is an index of the dynamic balance. For example, a score according to the estimation value of the FR distance (also referred to as a dynamic balance score) is an index of the dynamic balance. The dynamic balance score is a value obtained by scoring the FR distance, which is an index of the dynamic balance, on a preset basis. The dynamic balance is affected by attributes such as height. Therefore, the dynamic balance score may be scored with reference to each attribute. The index of the dynamic balance is not limited to the FR distance as long as the dynamic balance can be scored.

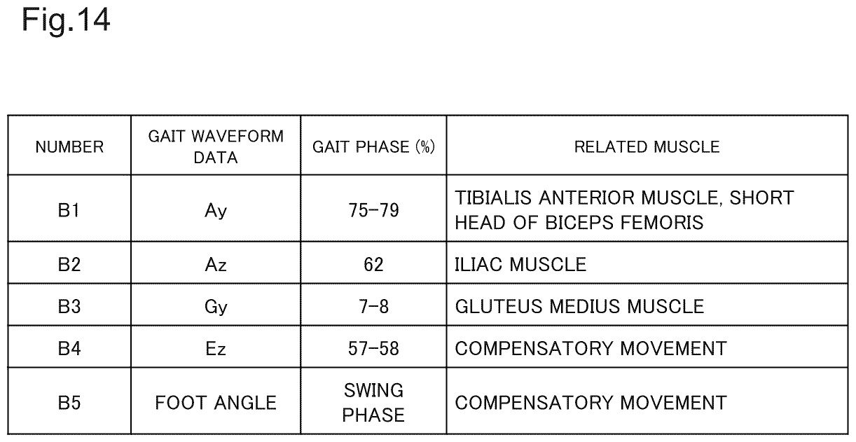

FIG. 14 is a correspondence table summarizing feature amounts used to estimate the dynamic balance. The correspondence table in FIG. 14 associates the number of the feature amount, the gait waveform data from which the feature amount is extracted, the gait phase (%) from which the gait phase cluster is extracted, and the related muscle. The FR distance is correlated with the activities of the gluteus medius muscle, the iliac muscle, the hamstrings (the long head of the biceps femoris), the tibialis anterior muscle, and the like, and the magnitude of the compensatory movement that makes the orientation of the toe outward. Therefore, feature amounts B1 to B5 extracted from the gait phase in which these features appear are used to estimate the FR distance.

The feature amount B1 is extracted from a section of gait phase 75 to 79% of gait waveform data Ay related to the time-series data of the traveling-direction acceleration (Y-direction acceleration). The gait phase 75 to 79% is included in the mid-swing period T6. The feature amount B1 mainly includes features regarding the motion of the tibialis anterior muscle and the short head of the biceps femoris.

The feature amount B2 is extracted from a section of gait phase 62% of gait waveform data Az related to the time-series data of the perpendicular-direction acceleration (Z-direction acceleration). The gait phase 62% is included in the initial swing period T5. The feature amount B2 mainly includes a feature regarding the motion of the iliac muscle.

The feature amount B3 is extracted from a section of gait phase 7 to 8% of gait waveform data Gy related to the time-series data of the angular velocity in the coronal plane (around the Y axis). The gait phase 7 to 8% is included in the load response period T1. The feature amount B3 mainly includes a feature regarding the motion of the gluteus medius muscle.

The feature amount B4 is extracted from a section of gait phase 57 to 58% of gait waveform data Ez related to the time-series data of the angle (attitude angle) in the horizontal plane (around the Z axis). The gait phase 57 to 58% is included in the pre-swing period T4. The feature amount B4 mainly includes a feature related to the compensatory movement. The compensatory movement is a movement of changing the foot angle to acquire stability in order to compensate for a decrease in balance ability and muscle function associated with aging.

The feature amount B5 is an average value of the foot angles in the horizontal plane in the swing phase. For example, the feature amount B5 is an average value in the swing phase of the gait waveform data Ez. In other words, the feature amount B5 is an integral value of the gait waveform data Gz related to the time-series data of the angular velocity in the horizontal plane (around the Z axis). The feature amount B5 mainly includes a feature related to the compensatory movement.

### <Related Item C>

The related item C relates to the lower-limb muscular strength. The lower-limb muscular strength can be evaluated by a result of a chair stand test. In the present example embodiment, the result of a 5-chair stand test in which chair standing and sitting is repeated five times is evaluated. The 5-chair stand test is also referred to as a Sit to Stand-5 (SS-5) test. The result of the 5-chair stand test is evaluated by the time for repeating chair standing and sitting five times (also referred to as standing and sitting time). The standing and sitting time is a result value of the SS-5 test. The shorter the standing and sitting time, the better the result of the SS-5 test. The result may be evaluated by the score of a 30-second chair stand (CS-30) test for measuring the number of times of chair standing and sitting movements for 30 seconds.

The index of the lower-limb muscular strength related to the related item C is the standing and sitting time. For example, an estimation value of the 5-standing and sitting time is an index of the lower-limb muscular strength. For example, a score according to the estimation value of the standing and sitting time (also referred to as a lower-limb muscular strength score) is an index of the lower-limb muscular strength. The lower-limb muscular strength score is a value obtained by scoring the standing and sitting time, which is an index of the lower-limb muscular strength, on a preset basis. The lower-limb muscular strength is affected by attributes such as age. Therefore, the lower-limb muscular strength score may be scored with reference to each attribute. The index of the lower-limb muscular strength is not limited to the standing and sitting time as long as the lower-limb muscular strength can be scored.

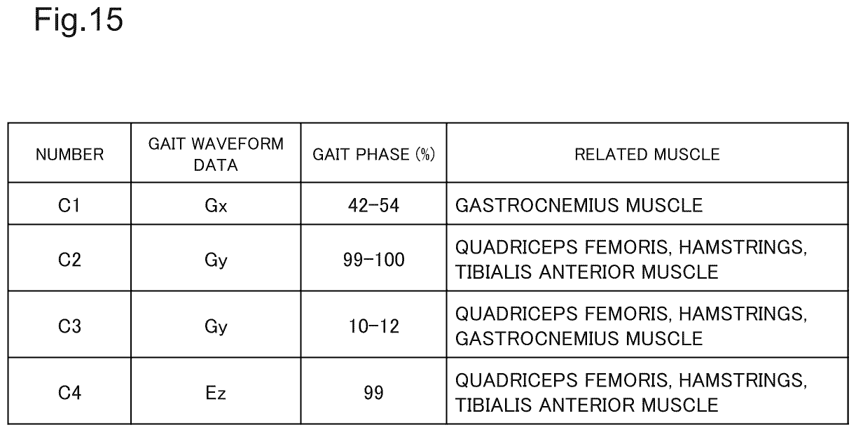

FIG. 15 is a correspondence table summarizing feature amounts used to estimate the lower-limb muscular strength. The correspondence table in FIG. 15 associates the number of the feature amount, the gait waveform data from which the feature amount is extracted, the gait phase (%) from which the gait phase cluster is extracted, and the related muscle. The standing and sitting time is correlated with the quadriceps femoris, the hamstrings, the tibialis anterior muscle, and the gastrocnemius muscle. Therefore, feature amounts C1 to C4 extracted from the gait phase in which these features appear are used to estimate the standing and sitting time.

The feature amount C1 is extracted from a section of gait phase 42 to 54% of gait waveform data Gx related to the time-series data of the angular velocity in the sagittal plane (around the X axis). The gait phase 42 to 54% is a section from the terminal stance period T3 to the pre-swing period T4. The feature amount C1 mainly includes a feature regarding the motion of the gastrocnemius muscle.

The feature amount C2 is extracted from a section of gait phase 99 to 100% of gait waveform data Gy related to the time-series data of the angular velocity in the coronal plane (around the Y axis). The gait phase 99 to 100% is the final phase of the terminal swing period T7. The feature amount C2 mainly includes features regarding the motion of the quadriceps femoris, the hamstrings, and the tibialis anterior muscle.

The feature amount C3 is extracted from a section of gait phase 10 to 12% of gait waveform data Gy related to the time-series data of the angular velocity in the coronal plane (around the Y axis). The gait phase 10 to 12% is the early phase of the mid-stance period T2. The feature amount C3 mainly includes features regarding the motion of the quadriceps femoris, the hamstrings, and the gastrocnemius muscle.

The feature amount C4 is extracted from a section of gait phase 99% of gait waveform data Ez related to the time-series data of the angle (attitude angle) in the horizontal plane (around the Z axis). The gait phase 99% is the final phase of the terminal swing period T7. The feature amount C4 mainly includes features regarding the motion of the quadriceps femoris, the hamstrings, and the tibialis anterior muscle.

### <Related Item D>

The related item D relates to the mobility. The mobility can be evaluated by a result of a time up and go (TUG) test. In the present example embodiment, the result of the TUG test is evaluated by the time (also referred to as TUG required time) from standing up from a chair, walking to a mark 3 m (meters) ahead, changing the direction, and sitting down again on the chair. The TUG required time is a result value of the TUG test. The shorter the TUG required time, the better the result of the TUG test. The related item D may be evaluated in terms of the result of test regarding the mobility other than the TUG test.

The index of the mobility regarding the related item D is the TUG required time. For example, an estimation value of the TUG required time is an index of the mobility. For example, a score according to the estimation value of the TUG required time (also referred to as a mobility score) is an index of the mobility. The mobility score is a value obtained by scoring the TUG required time, which is an index of the mobility, on a preset basis. The mobility is affected by attributes such as age. Therefore, the mobility score may be scored with reference to each attribute. The index of the mobility is not limited to the TUG required time as long as the mobility can be scored.

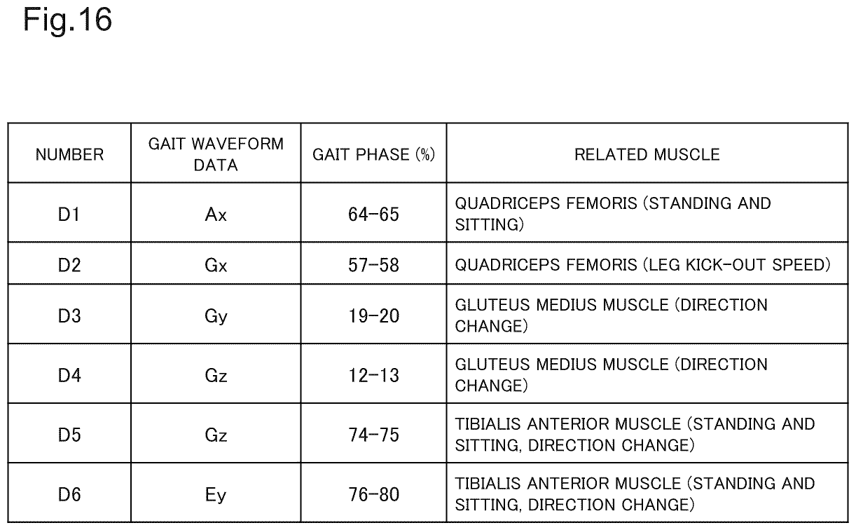

FIG. 16 is a correspondence table summarizing feature amounts used to estimate the mobility. The correspondence table in FIG. 16 associates the number of the feature amount, the gait waveform data from which the feature amount is extracted, the gait phase (%) from which the gait phase cluster is extracted, and the related muscle. The TUG required time is correlated with the quadriceps femoris, the gluteus medius muscle, and the tibialis anterior muscle. Therefore, feature amounts D1 to D6 extracted from the gait phase in which these features appear are used to estimate the TUG required time. The feature of the tensor fasciae latae muscle appears in the gait phases 0 to 45% and 85 to 100%. The feature of the gluteus medius muscle appears in the gait phase 0 to 25%. The feature of the tibialis anterior muscle appears in the gait phases 0 to 10% and 57 to 100%.

The feature amount D1 is extracted from a section of gait phase 64 to 65% of gait waveform data Ax related to the time-series data of the lateral-direction acceleration (X-direction acceleration). The gait phase 64 to 65% is included in the initial swing period T5. The feature amount D1 mainly includes features regarding the motion of the quadriceps femoris in the standing and sitting movement.

The feature amount D2 is extracted from a section of gait phase 57 to 58% of gait waveform data Gx related to the time-series data of the angular velocity in the sagittal plane (around the X axis). The gait phase 57 to 58% is included in the pre-swing period T4. The feature amount D2 mainly includes features regarding the motion of the quadriceps femoris related to a leg kick-out speed.

The feature amount D3 is extracted from a section of gait phase 19 to 20% of gait waveform data Gy related to the time-series data of the angular velocity in the coronal plane (around the Y axis). The gait phase 19 to 20% is included in the mid-stance period T2. The feature amount D3 mainly includes features regarding the motion of the gluteus medius muscle in changing the direction.

The feature amount D4 is extracted from a section of gait phase 12 to 13% of gait waveform data Ez related to the time-series data of the angular velocity in the horizontal plane (around the Z axis). The gait phase 12 to 13% is the early phase of the mid-stance period T2. The feature amount D4 mainly includes features regarding the motion of the gluteus medius muscle in changing the direction.

The feature amount D5 is extracted from a section of gait phase 74 to 75% of gait waveform data Ez related to the time-series data of the angular velocity in the horizontal plane (around the Z axis). The gait phase 74 to 75% is the early phase of the mid-swing period T6. The feature amount D5 mainly includes features regarding the motion of the tibialis anterior muscle in standing and sitting, and in changing the direction.

The feature amount D6 is extracted from a section of gait phase 76 to 80% of gait waveform data Ey related to the time-series data of the angle (attitude angle) in the coronal plane (around the Y axis). The gait phase 76 to 80% is included in the mid-swing period T6. The feature amount D6 mainly includes features regarding the motion of the tibialis anterior muscle in standing and sitting, and in changing the direction.

### <Related Item E>

The related item E relates to the static balance. The static balance can be evaluated by a result of a single leg standing test. In the present example embodiment, the result of the single leg standing test is evaluated by the time (also referred to as a single leg standing time) in which the eyes are closed and one leg is kept raised from the ground 5 cm (centimeters). The single leg standing time is a result value of the static balance. The longer the single leg standing time, the better the result of the static balance. The related item E may be evaluated by a result other than the eye-closed single leg standing test. For example, the related item E may be evaluated in a single leg standing test with the eyes are opened (eye-open single leg standing test) or in other variations of the single leg standing test.

The index of the static balance regarding the related item E is the single leg standing time. For example, the estimation value of the single leg standing time is an index of the static balance. For example, a score according to the estimation value of the single leg standing time (also referred to as a static balance score) is an index of the static balance. The static balance score is a value obtained by scoring the single leg standing time, which is an index of the static balance, on a preset basis. The static balance is affected by attributes such as age and height. Therefore, the static balance score may be scored with reference to each attribute. The index of the static balance is not limited to the single leg standing time as long as the static balance can be scored.

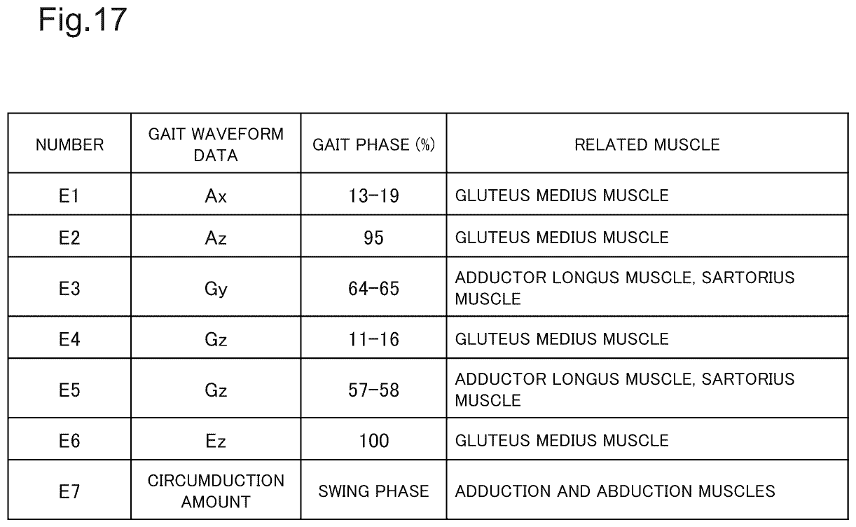

FIG. 17 is a correspondence table summarizing feature amounts used to estimate the static balance. The correspondence table in FIG. 17 associates the number of the feature amount, the gait waveform data from which the feature amount is extracted, the gait phase (%) from which the gait phase cluster is extracted, and the related muscle. The single leg standing time is correlated with the gluteus medius muscle, the adductor longus muscle, the sartorius muscle, and the adduction and abduction muscles. Therefore, feature amounts E1 to E7 extracted from the gait phase in which these features appear are used to estimate the single leg standing time.

The feature amount E1 is extracted from a section of gait phase 13 to 19% of gait waveform data Ax related to the time-series data of the lateral-direction acceleration (X-direction acceleration). The gait phase 13 to 19% is included in the mid-stance period T2. The feature amount E1 mainly includes a feature regarding the motion of the gluteus medius muscle.

The feature amount E2 is extracted from a section of gait phase 95% of gait waveform data Az related to the time-series data of the perpendicular-direction acceleration (Z-direction acceleration). The gait phase 95% is the final phase of the terminal swing period T7. The feature amount E2 mainly includes a feature regarding the motion of the gluteus medius muscle.

The feature amount E3 is extracted from a section of gait phase 64 to 65% of gait waveform data Gy related to the time-series data of the angular velocity in the coronal plane (around the Y axis). The gait phase 64 to 65% is included in the initial swing period T5. The feature amount E3 mainly includes features regarding the motion of the adductor longus muscle and the sartorius muscle.

The feature amount E4 is extracted from a section of gait phase 11 to 16% of gait waveform data Gz related to the time-series data of the angular velocity in the horizontal plane (around the Z axis). The gait phase 11 to 16% is included in the mid-stance period T2. The feature amount E4 mainly includes a feature regarding the motion of the gluteus medius muscle.

The feature amount E5 is extracted from a section of gait phase 57 to 58% of gait waveform data Gz related to the time-series data of the angular velocity in the horizontal plane (around the Z axis). The gait phase 57 to 58% is included in the pre-swing period T4. The feature amount E5 mainly includes features regarding the motion of the adductor longus muscle and the sartorius muscle.

The feature amount E6 is extracted from a section of gait phase 100% of gait waveform data Ez related to the time-series data of the angle (attitude angle) in the horizontal plane (around the Z axis). The gait phase 100% corresponds to the timing of heel contact when the terminal swing period T7 switches to the load response period T1. The feature amount of the gait waveform data Ez in the gait phase 100% corresponds to a foot angle in a state where the sole is grounded. The feature amount E6 mainly includes a feature regarding the motion of the gluteus medius muscle.

The feature amount E7 is a distance (circumduction amount) between the traveling axis and the foot at a timing when the central axis of the foot is farthest from the traveling axis in the swing phase. The feature amount E7 is a circumduction amount standardized by the height of the subject. The feature amount E7 mainly includes a feature regarding the motion of the adduction and abduction muscles.

### <Fall Probability>

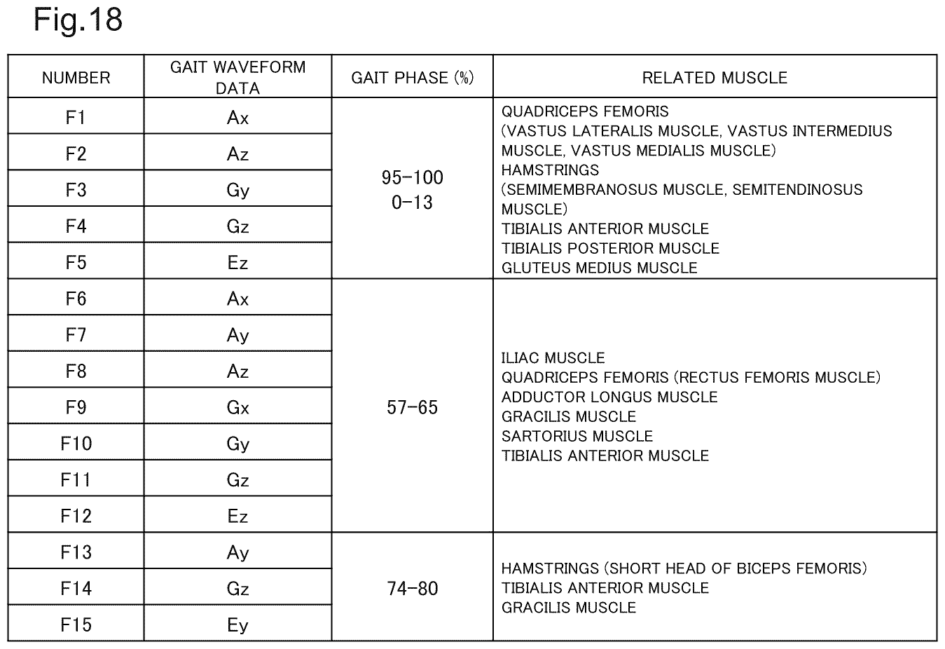

FIG. 18 is a correspondence table summarizing feature amounts used to estimate the fall probability. The correspondence table in FIG. 18 associates the number of the feature amount, the gait waveform data from which the feature amount is extracted, the gait phase (%) from which the gait phase cluster is extracted, and the related muscle. The fall probability can be estimated based on the features appearing in the gait phase common to the five items among the gait phases in which the features of the above-described five items appear. The gait phases common to the five items are a section immediately before the heel contact to immediately after the landing of the sole, a section before and after the toe off, and a section before and after the movement of minimum toe clearance in the mid-swing period. Therefore, feature amounts F1 to F15 extracted from these gait phases are used to estimate the fall probability.

The feature amounts F1 to F5 (also referred to as a first feature amount group) are extracted from the sections of the gait phases 0 to 13 and 95 to 100%. These sections correspond to a section (section of gait phase 95 to 13%) from the gait phase 95% of the preceding gait cycle to the gait phase 13% of the subsequent gait cycle. The gait phase 95 to 13% is a section (also referred to as a first section) from immediately before heel contact to immediately after the landing of the sole. The gait phase 95 to 13% extends from the terminal swing period T7 to the early phase of the mid-stance period T2. The feature amounts F1 to F5 include features regarding the motion of the quadriceps femoris (vastus lateralis muscle, vastus intermedius muscle, vastus medialis muscle), the hamstrings (semimembranosus muscle, semitendinosus muscle), the tibialis anterior muscle, the tibialis posterior muscle, and the gluteus medius muscle.

The feature amount F1 is extracted from gait waveform data Ax related to the time-series data of the lateral-direction acceleration (X-direction acceleration). The feature amount F2 is extracted from gait waveform data Az related to the time-series data of the perpendicular-direction acceleration (Z-direction acceleration). The feature amount F3 is extracted from gait waveform data Gy related to the time-series data of the angular velocity in the coronal plane (around the Y axis). The feature amount F4 is extracted from gait waveform data Gz related to the time-series data of the angular velocity in the horizontal plane (around the Z axis). The feature amount F5 is extracted from gait waveform data Ez related to the time-series data of the angle (attitude angle) in the horizontal plane (around the Z axis).

The feature amounts F6 to F12 (also referred to as a second feature amount group) are extracted from the section of the gait phase 57 to 65%. The gait phase 57 to 65% is a section (also referred to as a second section) before and after the toe off. The gait phase 57 to 65% extends from the pre-swing period T4 to the initial swing period T5. The feature amounts F6 to F12 include features regarding the motion of the iliopsoas muscle, the quadriceps femoris (rectus femoris muscle), the adductor longus muscle, the gracilis muscle, the sartorius muscle, and the tibialis anterior muscle.

The feature amount F6 is extracted from gait waveform data Ax related to the time-series data of the lateral-direction acceleration (X-direction acceleration). The feature amount F7 is extracted from gait waveform data Ay related to the time-series data of the traveling-direction acceleration (Y-direction acceleration). The feature amount F8 is extracted from gait waveform data Az related to the time-series data of the perpendicular-direction acceleration (Z-direction acceleration). The feature amount F9 is extracted from gait waveform data Gx related to the time-series data of the angular velocity in the sagittal plane (around the X axis). The feature amount F10 is extracted from gait waveform data Gy related to the time-series data of the angular velocity in the coronal plane (around the Y axis). The feature amount F11 is extracted from gait waveform data Gz related to the time-series data of the angular velocity in the horizontal plane (around the Z axis). The feature amount F12 is extracted from gait waveform data Ez related to the time-series data of the angle (attitude angle) in the horizontal plane (around the Z axis).

The feature amounts F13 to F15 (also referred to as a third feature amount group) are extracted from the section of the gait phase 74 to 80%. The gait phase 74 to 80% is a section (also referred to as a third section) before and after the movement of the minimum toe clearance in the mid-swing period. The gait phase 74 to 80% is included in the mid-swing period T6. The feature amounts F13 to F15 include features regarding the motion of the hamstrings (the short head of the biceps femoris), the tibialis anterior muscle, and the gracilis muscle.

The feature amount F13 is extracted from gait waveform data Ay related to the time-series data of the traveling-direction acceleration (Y-direction acceleration). The feature amount F14 is extracted from gait waveform data Gz related to the time-series data of the angular velocity in the horizontal plane (around the Z axis). The feature amount F15 is extracted from gait waveform data Ey related to the time-series data of the angle (attitude angle) in the coronal plane (around the Y axis).

### First Estimation Example

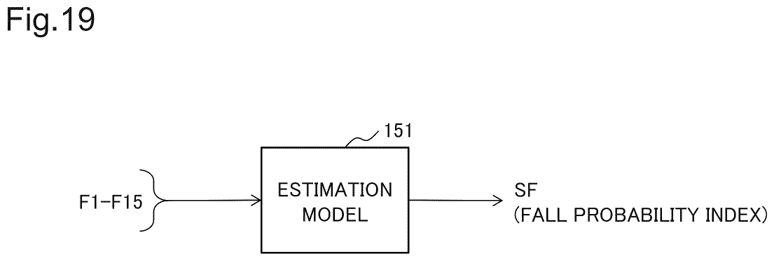

FIG. 19 is a conceptual diagram illustrating an example in which the feature amounts F1 to F15 extracted from the sensor data measured along with gait of the user are input to an estimation model 151 (first estimation model) constructed in advance for estimating the fall probability. The estimation model 151 outputs an estimation value of the fall probability index (fall probability score SF) according to the inputs of the feature amounts F1 to F15. For example, the estimation model 151 is generated by machine learning using training data in which the feature amounts F1 to F15 used for estimating the fall probability are explanatory variables and the fall probability is an objective variable. The estimation result of the estimation model 151 is not limited as long as the estimation result regarding the fall probability index (fall probability score SF) is output according to the input of the feature amount data for estimating the fall probability. For example, the estimation model 151 may be a model that estimates the fall probability using attribute data as an explanatory variable in addition to the feature amounts F1 to F15 used for estimating the fall probability.

For example, the storage unit 132 stores an estimation model for estimating the fall probability using a multiple regression prediction method. For example, the storage unit 132 stores a parameter for estimating the fall probability using Formula 1 described below.

$$Fall probability = f 1 \times F 1 + f 2 \times F 2 + … + f 15 \times F 15 + f 0 \qquad ( 1 )$$

In Formula 1 described above, F1, F2, . . . . F15 are feature amounts for each gait phase cluster used to estimate the fall probability indicated in the correspondence table in FIG. 18. In Formula 1 described above, F3 to F14 are omitted. f1, f2, . . . f15 are coefficients (weights) multiplied by F1, F2, . . . . F15. f0 is a constant term. In Formula 1 described above, f3 to f14 are omitted. For example, f0, f1, . . . f15 are stored in the storage unit 132.

### Second Estimation Example

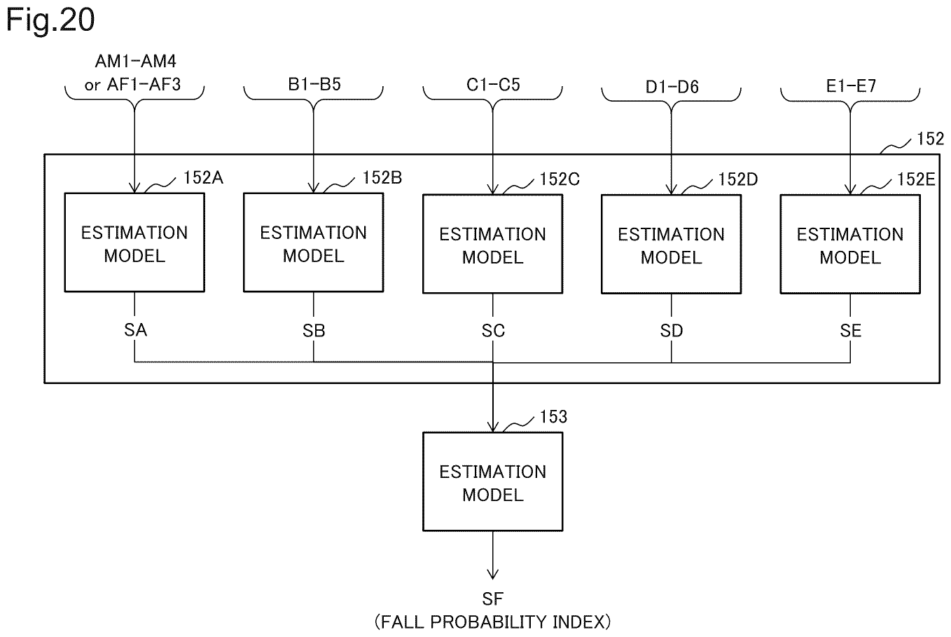

FIG. 20 is a conceptual diagram illustrating an example in which the output of an estimation model 152 (pre-estimation model) for estimating the scores of the five items is input to an estimation model 153 (second estimation model) constructed in advance for estimating the fall probability. The estimation model 152 includes an estimation model 152A, an estimation model 152B, an estimation model 152C, an estimation model 152D, and an estimation model 152E.

The estimation model 152A outputs a score (total muscular strength score SA) related to the total muscular strength (grip strength) of the whole body according to the inputs of the feature amounts AM1 to AM4 or the feature amounts AF1 to AF3 extracted from the sensor data measured along with the gait of the user. For example, the estimation model 152A may be a different model for males and for females. The estimation result of the estimation model 152A is not limited as long as the estimation result regarding the index of the total muscular strength is output according to the input of the feature amount data for estimating the total muscular strength. For example, the estimation model 152A may be a model that estimates the dynamic balance using attribute data such as age and height as explanatory variables in addition to the feature amounts AM1 to AM4 or the feature amounts AF1 to AF3.

For example, the storage unit 132 stores the estimation model 152A for estimating the total muscular strength score SA using a multiple regression prediction method. For example, the storage unit 132 stores a parameter for estimating the total muscular strength score SA using Formula 2 described below.

$$( 2 )$$

$$SA = m ( am 1 \times AM 1 + am 2 \times AM 2 + am 3 \times AM 3 + am 4 \times AM 4 + am 0 ) + f ( af 1 \times AF 1 + af 2 \times AF 2 + af 3 \times AF 3 + af 0 )$$

In Formula 2 described above, AM1, AM2, AM3, and AM4 are feature amounts for each gait phase cluster used to estimate the total muscular strength score SA of a male indicated in the correspondence table in FIG. 13. am1, am2, am3, and am4 are coefficients (weights) multiplied by AM1, AM2, AM3, and AM4. am0 is a constant term. AF1, AF2, and AF3 are feature amounts for each gait phase cluster used for estimation of the total muscular strength score SA of a female indicated in the correspondence table in FIG. 13. af1, af2, and af3 are coefficients (weights) multiplied by AF1, AF2, and AF3. af0 is a constant term. m and f are flags according to sex. In a case where the user is a male, m is 1 and f is 0. In a case where the user is a female, m is 0 and f is 1. For example, am0, am1, am2, am3, am4, af0, af1, af2, and af3 are stored in the storage unit 132.

The estimation model 152B outputs a score regarding the dynamic balance (dynamic balance score SB) according to the inputs of the feature amounts B1 to B5 extracted from the sensor data measured along with the gait of the user. The estimation result of the estimation model 152B is not limited as long as the estimation result regarding the index of the dynamic balance is output according to the input of the feature amount data for estimating the dynamic balance. For example, the estimation model 152B may be a model that estimates the dynamic balance using attribute data such as height as explanatory variables in addition to the feature amounts B1 to B5.

For example, the storage unit 132 stores the estimation model for estimating the dynamic balance score SB using a multiple regression prediction method. For example, the storage unit 132 stores a parameter for estimating the dynamic balance score SB using Formula 3 described below.

$$SB = b 1 \times B 1 + b 2 \times B 2 + b 3 \times B 3 + b 4 \times B 4 + b 5 \times B 5 + b 0 \qquad ( 3 )$$

In Formula 3 described above, B1, B2, B3, B4, and B5 are feature amounts for each gait phase cluster used to estimate the dynamic balance indicated in the correspondence table in FIG. 14. b1, b2, b3, b4, and b5 are coefficients (weights) multiplied by B1, B2, B3, B4, and B5. b0 is a constant term. For example, b0, b1, b2, b3, b4, and b5 are stored in the storage unit 132.

The estimation model 152C outputs a score regarding the lower-limb muscular strength (lower-limb muscular strength score SC) according to the inputs of the feature amounts C1 to C4 extracted from the sensor data measured along with the gait of the user. The estimation result of the estimation model 152C is not limited as long as the estimation result regarding the index of the lower-limb muscular strength is output according to the input of the feature amount data for estimating the lower-limb muscular strength. For example, the estimation model 152C may be a model that estimates the dynamic balance using attribute data such as age as explanatory variables in addition to the feature amounts C1 to C4.

For example, the storage unit 132 stores the estimation model for estimating the lower-limb muscular strength score SC using a multiple regression prediction method. For example, the storage unit 132 stores a parameter for estimating the lower-limb muscular strength score SC using Formula 4 described below.

$$SC = c 1 \times C 1 + c 2 \times C 2 + c 3 \times C 3 + c 4 \times C 4 + c 0 \qquad ( 4 )$$

In Formula 4 described above, C1, C2, C3, and C4 are feature amounts for each gait phase cluster used to estimate the lower-limb muscular strength indicated in the correspondence table in FIG. 15. c1, c2, c3, and c4 are coefficients (weights) multiplied by C1, C2, C3, and C4. c0 is a constant term. For example, c0, c1, c2, c3, and c4 are stored in the storage unit 132.

The estimation model 152D outputs a score regarding the mobility (mobility score SD) according to the inputs of the feature amounts D1 to D6 extracted from the sensor data measured along with the gait of the user. The estimation result of the estimation model 152D is not limited as long as the estimation result regarding the index of the mobility is output according to the input of the feature amount data for estimating the mobility. For example, the estimation model 152D may be a model that estimates the mobility using attribute data such as age as explanatory variables in addition to the feature amounts D1 to D6.

For example, the storage unit 132 stores the estimation model for estimating the mobility score SD using a multiple regression prediction method. For example, the storage unit 132 stores a parameter for estimating the mobility score SD using Formula 5 described below.

$$( 5 )$$

$$SD = d 1 \times D 1 + d 2 \times D 2 + d 3 \times D 3 + d 4 \times D 4 + d 5 \times D 5 + d 6 \times D 6 + d 0$$

In Formula 5 described above, D1, D2, D3, D4, D5, and D6 are feature amounts for each gait phase cluster used to estimate the mobility indicated in the correspondence table in FIG. 16. d1, d2, d3, d4, d5, and d6 are coefficients (weights) multiplied by D1, D2, D3, D4, D5, and D6. d0 is a constant term. For example, do, d1, d2, d3, d4, d5, and d6 are stored in the storage unit 132.

The estimation model 152E outputs a score regarding the static balance (static balance score SE) according to the inputs of the feature amounts E1 to E7 extracted from the sensor data measured along with the gait of the user. The estimation result of the estimation model 152E is not limited as long as the estimation result regarding the index of the static balance is output according to the input of the feature amount data for estimating the static balance. For example, the estimation model 152E may be a model that estimates the static balance using attribute data such as age and height as explanatory variables in addition to the feature amounts E1 to E7.

For example, the storage unit 132 stores an estimation model for estimating the static balance using a multiple regression prediction method. For example, the storage unit 132 stores a parameter for estimating the static balance using Formula 6 described below.

$$( 6 )$$

$$Single leg standing time = e 1 \times E 1 + e 2 \times E 2 + e 3 \times E 3 + e 4 \times E 4 + \\ e 5 \times E 5 + e 6 \times E 6 + e 7 \times E 7 + e 0$$

In Formula 6 described above, E1, E2, E3, E4, E5, E6, and E7 are feature amounts for each gait phase cluster used to estimate the static balance indicated in the correspondence table in FIG. 17. e1, e2, e3, e4, e5, e6, and e7 are coefficients (weights) multiplied by E1, E2, E3, E4, E5, E6, and E7. e0 is a constant term. For example, e0, e1, e2, e3, e4, e5, e6, and e7 are stored in the storage unit 132.

The estimation model 153 (second estimation model) outputs the score regarding the fall probability (fall probability score SF) according to the input of the score output from the estimation model 152. That is, the estimation model 153 outputs the fall probability score SF according to the input of the scores of the five items. At least any of the scores output from the estimation model 152A, the estimation model 152B, the estimation model 152C, the estimation model 152D, and the estimation model 152E is input to the estimation model 153. That is, it is sufficient if at least one of the scores output from the estimation model 152A, the estimation model 152B, the estimation model 152C, the estimation model 152D, and the estimation model 152E is input to the estimation model 153. As the number of scores input to the estimation model 153 is larger, the fall probability can be estimated with higher accuracy. The estimation result of the estimation model 153 is not limited as long as the estimation result regarding the fall probability is output according to the input of the score output from the estimation model 152. For example, the estimation model 153 may be a model that estimates the fall probability using attribute data as an explanatory variable in addition to the score output from the estimation model 152.

For example, the storage unit 132 stores an estimation model for estimating the fall probability score SF using a multiple regression prediction method. For example, the storage unit 132 stores a parameter for estimating the fall probability score SF using Formula 7 described below.

$$SF = sa \times SA + sb \times SB + sc \times SC + sd \times SD + se \times SE + sf \qquad ( 7 )$$

In Formula 7 described above, SA, SB, SC, SD, and SE are scores output from the estimation model 152A, the estimation model 152B, the estimation model 152C, the estimation model 152D, and the estimation model 152E included in the estimation model 152. sa, sb, sc, sd, and se are coefficients (weights) multiplied by SA, SB, SC, SD, and SE. sf is a constant term. For example, sa, sb, sc, sd, se, and sf are stored in the storage unit 132.

### (Operation)

Next, the operation of the fall probability estimation system 1 will be described with reference to the drawings. Hereinafter, the first estimation example (FIG. 19) and the second estimation example (FIG. 20) will be individually described. Hereinafter, the gait measurement device 10 and the fall probability estimation device 13 included in the fall probability estimation system 1 will be individually described. With respect to the gait measurement device 10, an operation of the feature amount data generation unit 12 included in the gait measurement device 10 will be described.

### First Estimation Example

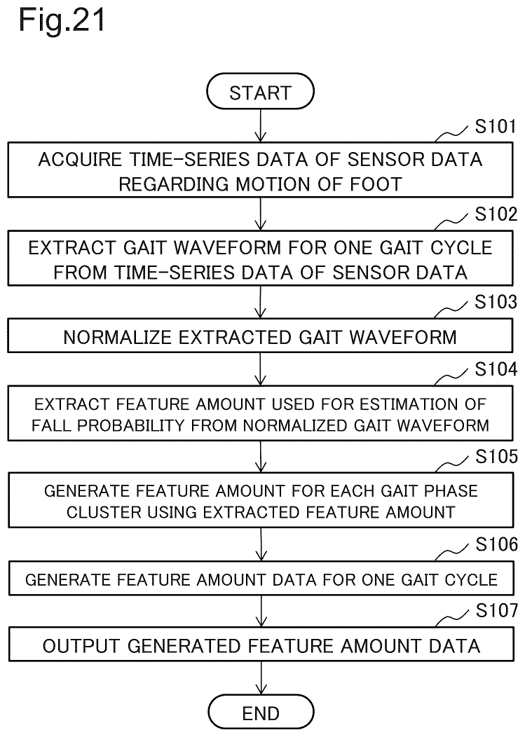

FIG. 21 is a flowchart for describing the operation of the feature amount data generation unit 12 included in the gait measurement device 10 in the first estimation example (FIG. 19). In the description along the flowchart of FIG. 21, the feature amount data generation unit 12 will be described as an operation subject.

In FIG. 21, first, the feature amount data generation unit 12 acquires the time-series data of the sensor data regarding the motion of the foot (Step S101).

Next, the feature amount data generation unit 12 extracts gait waveform data for one gait cycle from the time-series data of the sensor data (Step S102). The feature amount data generation unit 12 detects the heel contact and the toe off from the time-series data of the sensor data. The feature amount data generation unit 12 extracts the time-series data of the section between the consecutive pieces of heel contact as gait waveform data for one gait cycle.

Next, the feature amount data generation unit 12 normalizes the extracted gait waveform data for one gait cycle (Step S103). The feature amount data generation unit 12 normalizes the gait waveform data for one gait cycle to a gait cycle of 0 to 100% (first normalization). Further, the feature amount data generation unit 12 normalizes the ratio of the stance phase to the swing phase in the first-normalized gait waveform data for one gait cycle to 60:40 (second normalization).

Next, the feature amount data generation unit 12 extracts a feature amount from the gait phase used for estimation of the fall probability with respect to the normalized gait waveform (Step S104). For example, the feature amount data generation unit 12 extracts a feature amount input to an estimation model (first estimation model) constructed in advance.

Next, the feature amount data generation unit 12 generates a feature amount for each gait phase cluster using the extracted feature amount (Step S105).

Next, the feature amount data generation unit 12 integrates the feature amounts for each gait phase cluster to generate the feature amount data for one gait cycle (Step S106).

Next, the feature amount data generation unit 12 outputs the generated feature amount data to the fall probability estimation device 13 (Step S107).

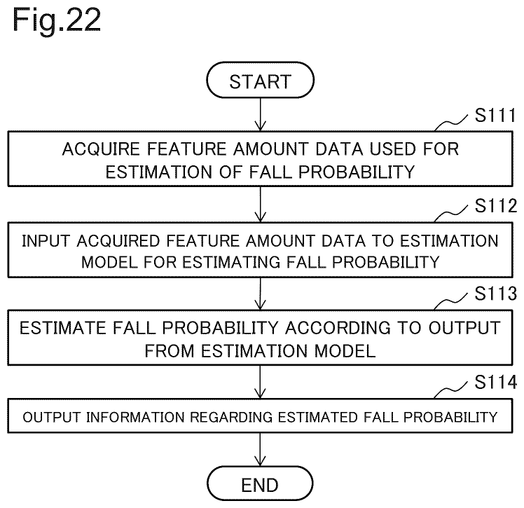

FIG. 22 is a flowchart for describing the operation of the fall probability estimation device 13 in the first estimation example (FIG. 19). In the description along the flowchart of FIG. 22, the fall probability estimation device 13 will be described as an operation subject.

In FIG. 22, first, the fall probability estimation device 13 acquires the feature amount data generated using the sensor data regarding the motion of the foot (Step S111).

Next, the fall probability estimation device 13 inputs the acquired feature amount data to the estimation model (first estimation model) for estimating the fall probability (single leg standing time) (Step S112).

Next, the fall probability estimation device 13 estimates the fall probability of the user according to the output (estimation value) from the estimation model (first estimation model) (Step S113). For example, the fall probability estimation device 13 estimates the single leg standing time of the user as the fall probability.

Next, the fall probability estimation device 13 outputs information regarding the estimated fall probability (Step S114). For example, the fall probability is output to a terminal device (not illustrated) carried by the user. For example, the fall probability is output to a system that executes processing using the fall probability.

### Second Estimation Example

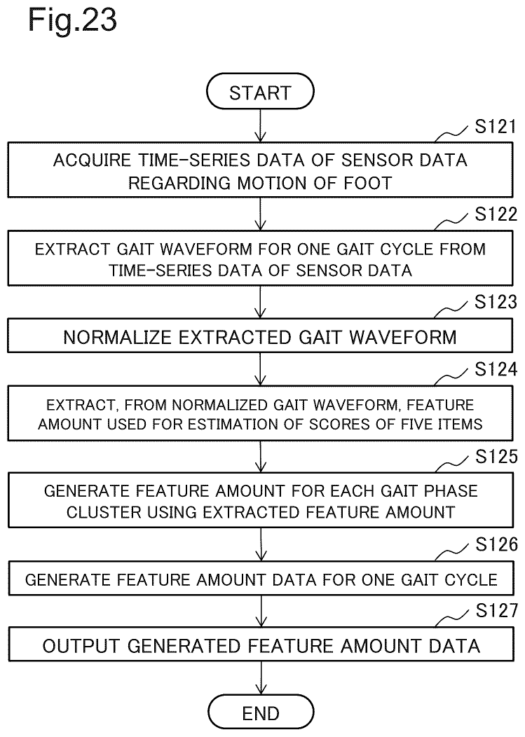

FIG. 23 is a flowchart for describing an operation of the feature amount data generation unit 12 included in the gait measurement device 10 in the second estimation example (FIG. 20). In the description along the flowchart of FIG. 23, the feature amount data generation unit 12 will be described as an operation subject.

In FIG. 23, first, the feature amount data generation unit 12 acquires the time-series data of the sensor data regarding the motion of the foot (Step S121).

Next, the feature amount data generation unit 12 extracts gait waveform data for one gait cycle from the time-series data of the sensor data (Step S122). The feature amount data generation unit 12 detects the heel contact and the toe off from the time-series data of the sensor data. The feature amount data generation unit 12 extracts the time-series data of the section between the consecutive pieces of heel contact as gait waveform data for one gait cycle.

Next, the feature amount data generation unit 12 normalizes the extracted gait waveform data for one gait cycle (Step S123). The feature amount data generation unit 12 normalizes the gait waveform data for one gait cycle to a gait cycle of 0 to 100% (first normalization). Further, the feature amount data generation unit 12 normalizes the ratio of the stance phase to the swing phase in the first-normalized gait waveform data for one gait cycle to 60:40 (second normalization).

Next, the feature amount data generation unit 12 extracts, from the normalized gait waveform, a feature amount used for estimation of the scores regarding the five items: the total muscular strength of the whole body; the dynamic balance, the lower-limb muscular strength; the mobility; and the static balance (Step S124). For example, the feature amount data generation unit 12 extracts a feature amount input to the estimation model (pre-estimation model) constructed in advance.

Next, the feature amount data generation unit 12 generates a feature amount for each gait phase cluster using the extracted feature amount (Step S125).

Next, the feature amount data generation unit 12 integrates the feature amounts for each gait phase cluster to generate the feature amount data for one gait cycle (Step S126).

Next, the feature amount data generation unit 12 outputs the generated feature amount data to the fall probability estimation device 13 (Step S127).

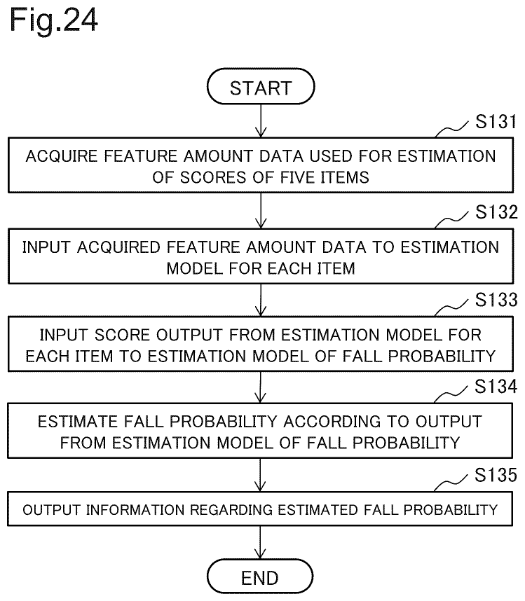

FIG. 24 is a flowchart for describing the operation of the fall probability estimation device 13 in the second estimation example (FIG. 20). In the description along the flowchart of FIG. 24, the fall probability estimation device 13 will be described as an operation subject.

In FIG. 24, first, the fall probability estimation device 13 acquires the feature amount data that is generated using the sensor data regarding the motion of the foot and used for estimation of the scores of the five items related to the fall probability (Step S131). The five items related to the fall probability are the total muscular strength of the whole body, the dynamic balance, the lower-limb muscular strength, the mobility, and the static balance.

Next, the fall probability estimation device 13 inputs the acquired feature amount data to the estimation model (pre-estimation model) including the estimation model for each item (Step S132).

Next, the fall probability estimation device 13 inputs the score output from the estimation model for each item included in the estimation model (pre-estimation model) to the estimation model (second estimation model) of the fall probability (Step S132).

Next, the fall probability estimation device 13 estimates the fall probability of the user according to the output (estimation value) from the estimation model (second estimation model) of the fall probability (Step S134). For example, the fall probability estimation device 13 estimates the fall probability score of the user using a total value of the scores output from the estimation models for the items. For example, the fall probability estimation device 13 estimates the fall probability score of the user using an average value of the scores output from the estimation models for the items. For example, the fall probability estimation device 13 estimates the fall probability score of the user by weighting, for each item, the scores output from the estimation models for the items.

Next, the fall probability estimation device 13 outputs information regarding the estimated fall probability (Step S135). For example, the fall probability is output to a terminal device (not illustrated) carried by the user. For example, the fall probability is output to a system that executes processing using the fall probability.

### Application Example

Next, an application example according to the present example embodiment will be described with reference to the drawings. In the application example described below, an example in which the function of the fall probability estimation device 13 installed in the mobile terminal carried by the user estimates information regarding the fall probability using the feature amount data measured by the gait measurement device 10 arranged in a shoe will be described.

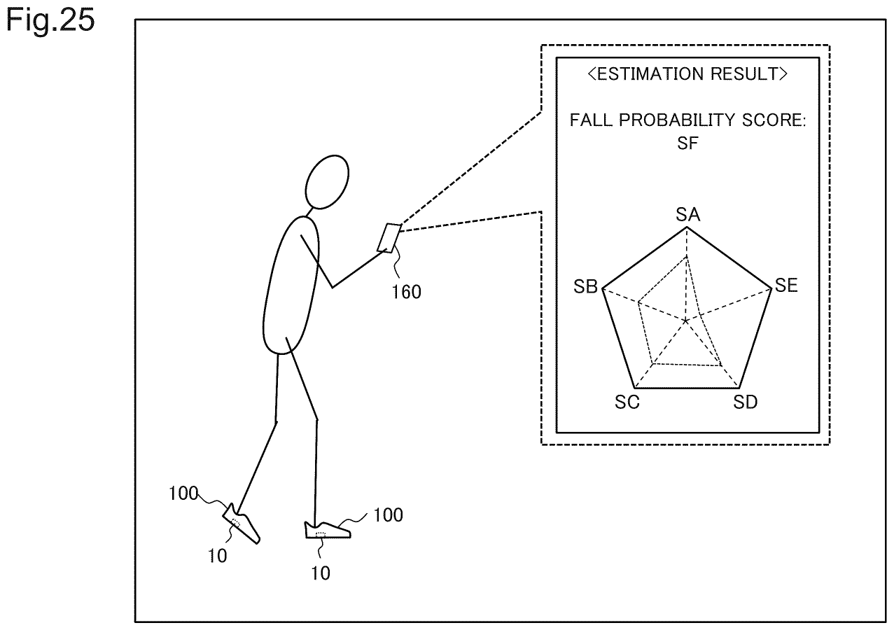

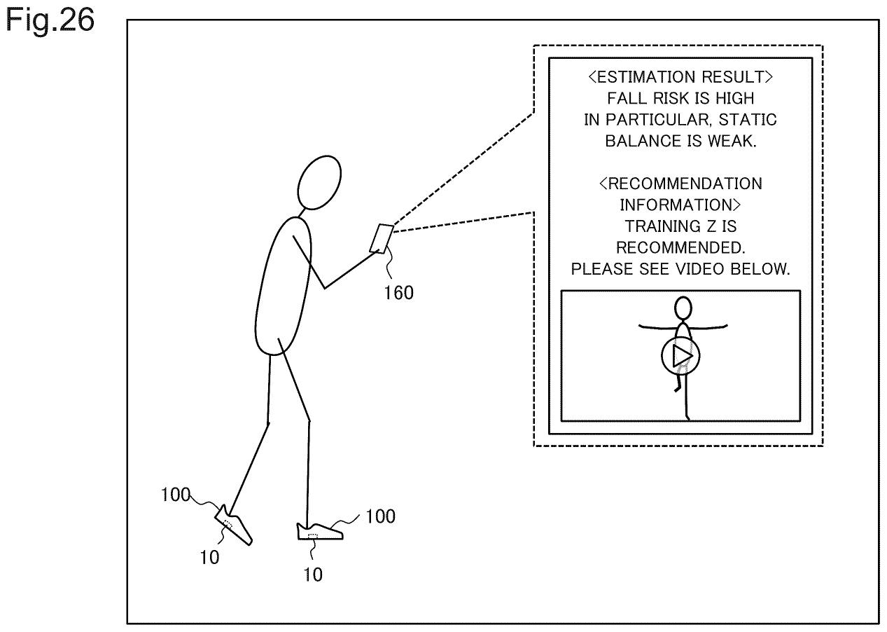

FIGS. 25 to 26 are conceptual diagrams illustrating an example in which the estimation result by the fall probability estimation device 13 is displayed on the screen of a mobile terminal 160 carried by the user walking while wearing the shoe 100 in which the gait measurement device 10 is arranged. FIGS. 25 to 26 are examples in which information according to the estimation result of the fall probability using the feature amount data according to the sensor data measured during gait of the user is displayed on the screen of the mobile terminal 160.

FIG. 25 is an example in which the fall probability score SF according to the estimation result of the fall probability is displayed on the screen of the mobile terminal 160. In the example of FIG. 25, a radar chart according to the value of the score used to estimate the fall probability score SF is displayed on the screen of the mobile terminal 160. The user who has confirmed the value of the fall probability score SF displayed on a display unit of the mobile terminal 160 can recognize its own fall probability according to the value of the fall probability score SF. In the example of FIG. 25, the score of the static balance score SE is low. The user who has confirmed the radar chart displayed on the display unit of the mobile terminal 160 can recognize that its own static balance score SE is low. The information regarding the estimated fall probability may be provided to other than the user. For example, the information regarding the estimated fall probability may be output to a trainer who manages the physical condition of the user or a terminal device (not illustrated) used by a family of the user, or the like. For example, the information regarding the estimated fall probability may be recorded in a database (not illustrated) constructed for the purpose of health management or the like.

FIG. 26 is another example in which the information regarding the fall probability according to the estimation result of the fall probability is displayed on the screen of the mobile terminal 160. In the example of FIG. 26, information regarding the fall probability “Fall risk is high” is displayed on the screen of the mobile terminal 160 according to the estimation result of the fall probability. In the example of FIG. 26, information regarding the static balance the result of which is low among the scores of the five items used for the estimation of the fall probability is displayed on the display unit of the mobile terminal 160. In the example of FIG. 26, information regarding the static balance “Static balance is low” is displayed on the display unit of the mobile terminal 160 according to the estimation value of the static balance score SE. Further, in the example of FIG. 26, recommendation information according to the estimation result of the static balance “Training Z is recommended. Please see the video below” is displayed on the display unit of the mobile terminal 160 according to the estimation value of the static balance score SE. The user who has confirmed the information displayed on the display unit of the mobile terminal 160 can practice training leading to an increase in static balance by exercising with reference to the video of the training Z according to the recommendation information.

As described above, the fall probability estimation system of the present example embodiment includes the gait measurement device and the fall probability estimation device. The gait measurement device includes the sensor and the feature amount data generation unit. The sensor includes the acceleration sensor and the angular velocity sensor. The sensor measures a spatial acceleration using the acceleration sensor. The sensor measures a spatial angular velocity using the angular velocity sensor. The sensor generates sensor data regarding the motion of the foot by using the measured spatial acceleration and spatial angular velocity. The sensor outputs the generated sensor data to the feature amount data generation unit. The feature amount data generation unit acquires the time-series data of the sensor data regarding the motion of the foot. The feature amount data generation unit extracts the gait waveform data for one gait cycle from the time-series data of the sensor data. The feature amount data generation unit normalizes the extracted gait waveform data. The feature amount data generation unit extracts, from the normalized gait waveform data, a feature amount used to estimate the fall probability from the gait phase cluster including at least one temporally continuous gait phase. The feature amount data generation unit generates the feature amount data including the extracted feature amount. The feature amount data generation unit outputs the generated feature amount data.

The fall probability estimation device includes the data acquisition unit, the storage unit, the estimation unit, and the output unit. The data acquisition unit acquires the feature amount data including the feature amount extracted from the sensor data regarding the motion of the foot of the user and used for estimating the fall probability of the user. The storage unit stores the estimation model that outputs the fall probability index according to the input of the feature amount data. The estimation unit inputs the acquired feature amount data to the estimation model. The estimation unit estimates the fall probability of the user according to the fall probability index output from the estimation model. The output unit outputs information regarding the estimated fall probability.

The fall probability estimation system of the present example embodiment estimates the fall probability of the user using the feature amount extracted from the sensor data regarding the motion of the foot of the user. Therefore, with the fall probability estimation system of the present example embodiment, the fall probability can be appropriately estimated in everyday life without using an instrument for measuring the fall probability.

In one aspect of the present example embodiment, the data acquisition unit acquires the feature amount data including the feature amount extracted from the gait waveform data generated using the time-series data of the sensor data regarding the motion of the foot and used to estimate the fall probability score as the fall probability index. The storage unit stores the estimation model that outputs the fall probability score according to the input of the feature amount data. The estimation unit inputs the acquired feature amount data to the estimation model, and estimates the fall probability of the user according to the fall probability score output from the estimation model. According to the present aspect, the fall probability can be appropriately estimated according to the fall probability score estimated using the sensor data regarding the motion of the foot.

In one aspect of the present example embodiment, the storage unit stores an estimation model generated by machine learning using training data regarding a plurality of subjects in which a feature amount used for estimation of the fall probability index is an explanatory variable and a fall probability index of the plurality of subjects is an objective variable. The estimation unit inputs the feature amount data acquired regarding the user to the estimation model, and estimates the fall probability of the user according to the fall probability index of the user output from the estimation model. According to the present aspect, the fall probability can be appropriately estimated using the estimation model in which the training data related to the plurality of subjects is machine-learned.

In one aspect of the present example embodiment, the storage unit stores an estimation model machine-learned using explanatory variables including attribute data of a plurality of subjects. The estimation unit inputs the feature amount data and the attribute data regarding the user to the estimation model, and estimates the fall probability of the user according to the fall probability index of the user output from the estimation model. In the present aspect, the fall probability is estimated by including attribute data that affects the fall probability. Therefore, according to the present aspect, the fall probability can be measured with higher accuracy according to the attribute of the user.

In one aspect of the present example embodiment, the storage unit stores the first estimation model generated by machine learning using training data for the gait waveform data of a plurality of subjects in which the feature amount regarding the muscle activity extracted from a specific section is an explanatory variable and the fall probability index is an objective variable. The specific section includes at least one section of the first section, the second section, and the third section. The first section is a section from immediately before the heel contact to immediately after the landing of the sole. From the first section, the feature amounts related to the activities of the quadriceps femoris, the hamstrings, the tibialis anterior muscle, the tibialis posterior muscle, and the gluteus medius muscle are extracted. The second section is a section from the pre-swing period to the initial swing period. From the second section, the feature amounts related to the activities of the iliopsoas muscle, the quadriceps femoris, the adductor longus muscle, the gracilis muscle, the sartorius muscle, and the tibialis anterior muscle are extracted. The third section is a section before and after the movement of the minimum toe clearance in the mid-swing period. From the third section, the feature amounts related to the activities of the hamstrings, the tibialis anterior muscle, and the gracilis muscle are extracted. The estimation unit inputs the feature amount data acquired according to the gait of the user to the first estimation model, and estimates the fall probability of the user according to the fall probability index of the user output from the first estimation model. According to the present aspect, it is possible to estimate the fall probability more matching with the physical activity by using the estimation model in which the feature amount according to the muscle activity that affects the fall probability is machine-learned.

In one aspect of the present example embodiment, the storage unit stores the first estimation model generated by machine learning using training data in which at least one feature amount included in the first feature amount group, the second feature amount group, and the third feature amount group is an explanatory variable and the fall probability index is an objective variable. The first feature amount group includes at least one feature amount extracted from the first section. The second feature amount group includes at least one feature amount extracted from the second section. The third feature amount group includes at least one feature amount extracted from the third section. The data acquisition unit acquires feature amount data including at least one feature amount included in the first feature amount group, the second feature amount group, and the third feature amount group extracted according to the gait of the user. The estimation unit inputs the acquired feature amount data to the first estimation model, and estimates the fall probability of the user according to the fall probability index of the user output from the first estimation model. According to the present aspect, it is possible to estimate the fall probability more matching with the physical activity by using the feature amount extracted from the gait phase from which the feature amount according to the muscle activity that affects the fall probability is extracted.

In one aspect of the present example embodiment, the storage unit stores the first estimation model generated by machine learning using training data in which the feature amount extracted from the gait phase common to the five items related to the fall probability is an explanatory variable and the fall probability index of the plurality of subjects is an objective variable. The five items are the total muscular strength of the whole body, the dynamic balance, the lower-limb muscular strength, the mobility, and the static balance. The data acquisition unit acquires feature amount data including at least one feature amount included in the first feature amount group, the second feature amount group, and the third feature amount group extracted according to the gait of the user. The estimation unit inputs the acquired feature amount data to the first estimation model, and estimates the fall probability of the user according to the fall probability index of the user output from the first estimation model. In the present aspect, the estimation model is used in which the feature amount extracted from the gait waveform data including a feature according to the muscle activity that affects the fall probability is machine-learned. Therefore, according to the present aspect, the fall probability more matching with the physical activity can be estimated.

In one aspect of the present example embodiment, the storage unit stores the second estimation model generated by machine learning using training data in which the score of at least one of the five items estimated using the gait waveform data of the plurality of subjects is an explanatory variable and the fall probability index is an objective variable. The data acquisition unit acquires at least one of the scores regarding the five items estimated according to the gait of the user. The estimation unit inputs the acquired score to the second estimation model, and estimates the fall probability of the user according to the fall probability index of the user output from the second estimation model. According to the present aspect, the fall probability can be appropriately estimated using the scores related to the five items.

In one aspect of the present example embodiment, the storage unit stores the pre-estimation model generated by machine learning using training data in which the feature amount related to at least one of the five items is an explanatory variable and the scores of the five items related to the feature amount used as an explanatory variable is an objective variable. The data acquisition unit acquires the feature amount regarding at least one of the five items extracted according to the gait of the user. The estimation unit inputs the acquired feature amount to the pre-estimation model, inputs the score output from the pre-estimation model to the second estimation model, and estimates the fall probability of the user according to the fall probability index of the user output from the second estimation model. According to the present aspect, the fall probability can be appropriately estimated using the scores related to the five items estimated using the sensor data regarding the motion of the foot.

In one aspect of the present example embodiment, the fall probability estimation device is mounted on a terminal device having a screen visually recognizable by the user. For example, the fall probability estimation device displays information regarding the fall probability estimated according to the sensor data regarding the motion of the foot of the user on the screen of the terminal device. For example, the fall probability estimation device displays recommendation information according to the fall probability estimated according to the sensor data regarding the motion of the foot of the user on the screen of the terminal device. For example, the fall probability estimation device displays, on the screen of the terminal device, a video related to training for building up a body part related to the fall probability as recommendation information according to the fall probability estimated according to the sensor data regarding the motion of the foot of the user. According to the present aspect, by displaying the fall probability estimated according to the feature amount extracted from the sensor data regarding the motion of the foot of the user on the screen visually recognizable by the user, the user can confirm the information according to its own fall probability.

### Second Example Embodiment

Next, a machine learning system according to a second example embodiment will be described with reference to the drawings. The machine learning system of the present example embodiment generates an estimation model for estimating the fall probability according to the input of the feature amount by machine learning using the feature amount data extracted from the sensor data measured by the gait measurement device.

### (Configuration)

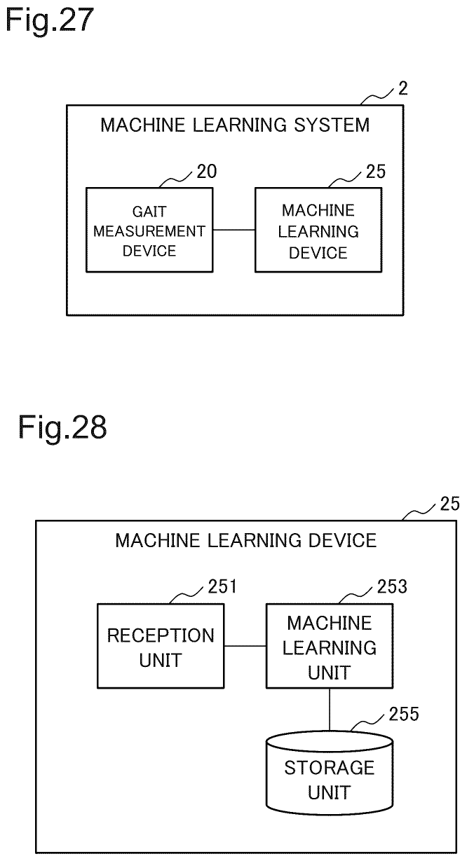

FIG. 27 is a block diagram illustrating an example of a configuration of a machine learning system 2 according to the present example embodiment. The machine learning system 2 includes a gait measurement device 20 and a machine learning device 25. The gait measurement device 20 and the machine learning device 25 may be connected by wire or wirelessly. The gait measurement device 20 and the machine learning device 25 may be configured by a single device. The machine learning system 2 may include only the machine learning device 25 by excluding the gait measurement device 20 from the configuration of the machine learning system 2. Although only one gait measurement device 20 is illustrated in FIG. 27, one gait measurement device 20 may be arranged on each of the left and right feet (two in total). The machine learning device 25 may be configured not to be connected to the gait measurement device 20 but to execute machine learning using the feature amount data generated in advance by the gait measurement device 20 and stored in a database.

The gait measurement device 20 is installed on at least one of the left and right feet. The gait measurement device 20 has the same configuration as the gait measurement device 10 of the first example embodiment. The gait measurement device 20 includes an acceleration sensor and an angular velocity sensor. The gait measurement device 20 converts the measured physical quantity into digital data (also referred to as sensor data). The gait measurement device 20 generates normalized gait waveform data for one gait cycle from time-series data of the sensor data. The gait measurement device 20 generates feature amount data used for estimating the fall probability. For example, the gait measurement device 20 generates feature amount data used for estimating the fall probability score SF. For example, the gait measurement device 20 generates feature amount data used for estimation of the scores regarding the five items: the total muscular strength of the whole body; the dynamic balance; the lower-limb muscular strength: the mobility; and the static balance. The gait measurement device 20 transmits the generated feature amount data to the machine learning device 25. The gait measurement device 20 may be configured to transmit the feature amount data to a database (not illustrated) accessed by the machine learning device 25. The feature amount data accumulated in the database is used for machine learning of the machine learning device 25.

The machine learning device 25 receives the feature amount data from the gait measurement device 20. When using the feature amount data accumulated in the database (not illustrated), the machine learning device 25 receives the feature amount data from the database. The machine learning device 25 executes machine learning using the received feature amount data. For example, the machine learning device 25 machine-learns training data in which the feature amount data extracted the gait waveform data of a plurality of subjects is an explanatory variable and the fall probability score SF according to the feature amount data is an objective variable. For example, the machine learning device 25 machine-learns training data in which the feature amount data extracted the gait waveform data of a plurality of subjects is an explanatory variable and the scores of the five items according to the feature amount data are objective variables. For example, the machine learning device 25 machine-learns training data in which at least one of the scores of the five items is an explanatory variable and the fall probability score SF according to the score is an objective variable. The machine learning algorithm executed by the machine learning device 25 is not particularly limited. The machine learning device 25 generates an estimation model machine-learned using training data related to a plurality of subjects. The machine learning device 25 stores the generated estimation model. The estimation model machine-learned by the machine learning device 25 may be stored in a storage device outside the machine learning device 25.

### [Machine Learning Device]

Next, details of the machine learning device 25 will be described with reference to the drawings. FIG. 28 is a block diagram illustrating an example of a detailed configuration of the machine learning device 25. The machine learning device 25 includes a reception unit 251, a machine learning unit 253, and a storage unit 255.

The reception unit 251 receives the feature amount data from the gait measurement device 20. The reception unit 251 outputs the received feature amount data to the machine learning unit 253. The reception unit 251 may receive the feature amount data from the gait measurement device 20 via a wire such as a cable, or may receive the feature amount data from the gait measurement device 20 via wireless communication. For example, the reception unit 251 is configured to receive the feature amount data from the gait measurement device 20 via a wireless communication function (not illustrated) conforming to a standard such as Bluetooth (registered trademark) or Wi-Fi (registered trademark). The communication function of the reception unit 251 may conform to a standard other than Bluetooth (registered trademark) or Wi-Fi (registered trademark).

The machine learning unit 253 acquires the feature amount data from the reception unit 251. The machine learning unit 253 executes machine learning using the acquired feature amount data. For example, the machine learning unit 253 machine-learns a data set in which the feature amount data extracted from the sensor data measured according to the motion of the foot of the subject is an explanatory variable and the fall probability score SF of the subject is an objective variable as training data. For example, the machine learning unit 253 machine-learns a data set in which the feature amount data extracted from the sensor data measured according to the motion of the foot of the subject is an explanatory variable and the scores of the five items of the subject are objective variables as training data. For example, the machine learning unit 253 machine-learns training data in which at least one of the scores of the five items is an explanatory variable and the fall probability score SF according to the score is an objective variable. For example, the machine learning unit 253 generates an estimation model according to the attribute data. For example, the machine learning unit 253 generates an estimation model for estimating the fall probability score SF using the feature amount data extracted from the sensor data measured according to the motion of the foot of the subject and the attribute data of the subject as explanatory variables. The machine learning unit 253 causes the storage unit 255 to store an estimation model machine-learned regarding a plurality of subjects.

For example, the machine learning unit 253 executes machine learning using a linear regression algorithm. For example, the machine learning unit 253 executes machine learning using an algorithm of a support vector machine (SVM). For example, the machine learning unit 253 executes machine learning using an algorithm of Gaussian process regression (GPR). For example, the machine learning unit 253 executes machine learning using an algorithm of random forest (RF). For example, the machine learning unit 253 may execute unsupervised machine learning of classifying a subject who is a generation source of the feature amount data according to the feature amount data. The machine learning algorithm executed by the machine learning unit 253 is not particularly limited.

The machine learning unit 253 may execute machine learning using the gait waveform data for one gait cycle as an explanatory variable. For example, the machine learning unit 253 executes supervised machine learning in which the gait waveform data of the accelerations in the three axial directions, the angular velocities around the three axes, and the angles (attitude angles) around the three axes is an explanatory variable and a correct value of the fall probability index is an objective variable. For example, in a case where the gait phase is set in increments of 1% in a gait cycle of 0 to 100%, the machine learning unit 253 machine-learns by using 909 explanatory variables.

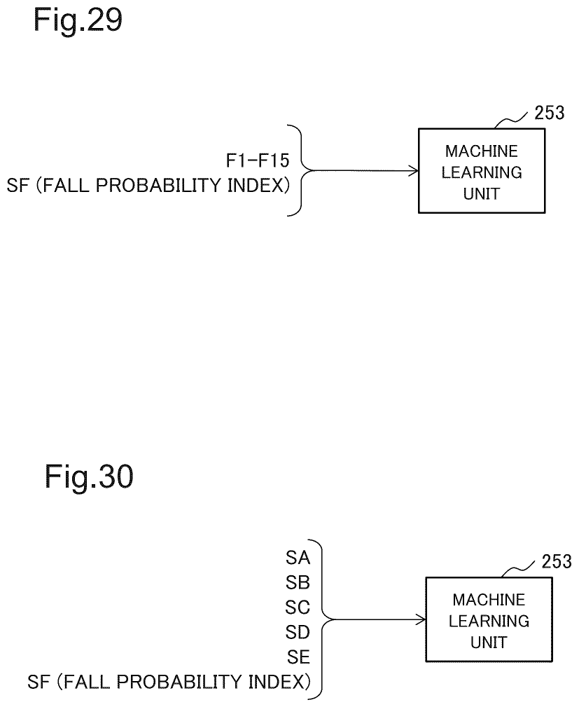

FIG. 29 is a conceptual diagram for describing machine learning for generating an estimation model (first estimation model). FIG. 29 is a conceptual diagram illustrating an example of causing the machine learning unit 253 to machine-learn a data set of the feature amounts F1 to F15 that are explanatory variables and the fall probability score SF (fall probability index) that is an objective variable as training data. For example, the machine learning unit 253 machine-learns data related to a plurality of subjects, and generates an estimation model (first estimation model) that outputs an output (estimation value) related to the fall probability score SF (fall probability index) according to the input of the feature amount extracted from the sensor data.

FIG. 30 is a conceptual diagram for describing machine learning for generating an estimation model (second estimation model). FIG. 30 is a conceptual diagram illustrating an example of causing the machine learning unit 253 to machine-learn a data set of the scores of the five items that are explanatory variables and the fall probability score SF (fall probability index) that is an objective variable as training data. In the example of FIG. 30, training data is used in which the total muscular strength score SA of the whole body, the dynamic balance score SB, the lower-limb muscular strength score SC, the mobility score SD, and the static balance score SE are explanatory variables and the fall probability score SF (fall probability index) is an objective variable. For example, the machine learning unit 253 machine-learns data related to a plurality of subjects, and generates an estimation model (second estimation model) that outputs an estimation value related to the fall probability score SF (fall probability index) according to the input of the feature amount extracted from the sensor data. Detailed description of the machine learning for generating the pre-estimation model is omitted. In the machine learning for generating the pre-estimation model, a plurality of estimation models included in the pre-estimation model may be individually generated, or a plurality of estimation models may be collectively generated.

The storage unit 255 stores an estimation model for estimating the fall probability machine-learned regarding a plurality of subjects. The estimation model stored in the storage unit 255 is used for the estimation of the fall probability by the fall probability estimation device 13 of the first example embodiment.

As described above, the machine learning system of the present example embodiment includes the gait measurement device and the machine learning device. The gait measurement device acquires the time-series data of the sensor data regarding the motion of the foot. The gait measurement device extracts the gait waveform data for one gait cycle from the time-series data of the sensor data, and normalizes the extracted gait waveform data. The gait measurement device extracts, from the normalized gait waveform data, a feature amount used to estimate the fall probability of the user from the gait phase cluster including at least one temporally continuous gait phase. The gait measurement device generates the feature amount data including the extracted feature amount. The gait measurement device outputs the generated feature amount data to the machine learning device.

The machine learning device includes a reception unit, a machine learning unit, and a storage unit. The reception unit acquires the feature amount data generated by the gait measurement device. The machine learning unit executes machine learning using the feature amount data. The machine learning unit generates an estimation model (first estimation model) that outputs the fall probability according to the input of the feature amount (second feature amount) of the gait phase cluster extracted from the time-series data of the sensor data measured along with the gait of the user. The estimation model (first estimation model) generated by the machine learning unit is stored in the storage unit.

The machine learning system of the present example embodiment generates an estimation model (first estimation model) using the feature amount data measured by the gait measurement device. Therefore, according to the present aspect, it is possible to generate an estimation model capable of appropriately estimating the fall probability in everyday life without using an instrument for measuring the fall probability.

Next, in one aspect of the present example embodiment, the gait measurement device extracts, from the normalized gait waveform data, a feature amount related to at least one of the five items: the total muscular strength of the whole body; the dynamic balance; the lower-limb muscular strength: the mobility; and the static balance. For example, the machine learning unit generates the estimation model (pre-estimation model) by machine learning using training data in which the feature amount related to at least one of the five items is an explanatory variable and the scores of the five items related to the feature amount used as an explanatory variable is an objective variable. The machine learning unit generates an estimation model (second estimation model) that outputs the fall probability index according to the input of the score related to at least one of the five items. According to the present aspect, it is possible to generate an estimation model capable of appropriately estimating the fall probability according to the input of the score related to the five items.

### Third Example Embodiment

Next, a fall probability estimation device according to a third example embodiment will be described with reference to the drawings. The fall probability estimation device of the present example embodiment has a simplified configuration of the fall probability estimation device included in the fall probability estimation system of the first example embodiment.

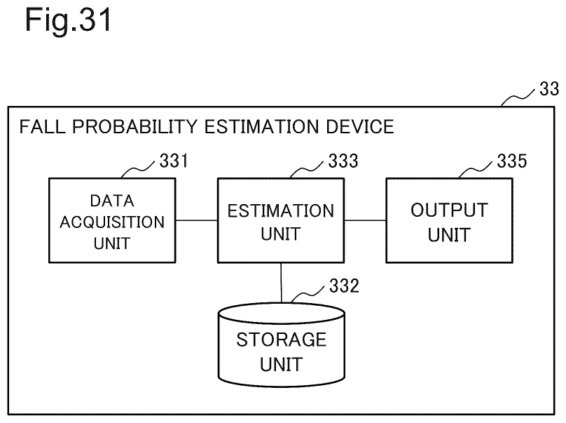

FIG. 31 is a block diagram illustrating an example of a configuration of a fall probability estimation device 33 according to the present example embodiment. The fall probability estimation device 33 includes a data acquisition unit 331, a storage unit 332, an estimation unit 333, and an output unit 335.

The data acquisition unit 331 acquires the feature amount data including the feature amount extracted from the sensor data regarding the motion of the foot of the user and used for estimating the fall probability index of the user. The storage unit 332 stores the estimation model that outputs the fall probability index according to the input of the feature amount data. The estimation unit 333 inputs the acquired feature amount data to the estimation model to estimate the fall probability of the user according to the fall probability index output from the estimation model. The output unit 335 outputs information regarding the estimated fall probability.

As described above, according to the present example embodiment, the fall probability of the user is estimated using the feature amount extracted from the sensor data regarding the motion of the foot of the user. Therefore, according to the present example embodiment, the fall probability can be appropriately estimated in everyday life without using an instrument for measuring the fall probability.

### (Hardware)

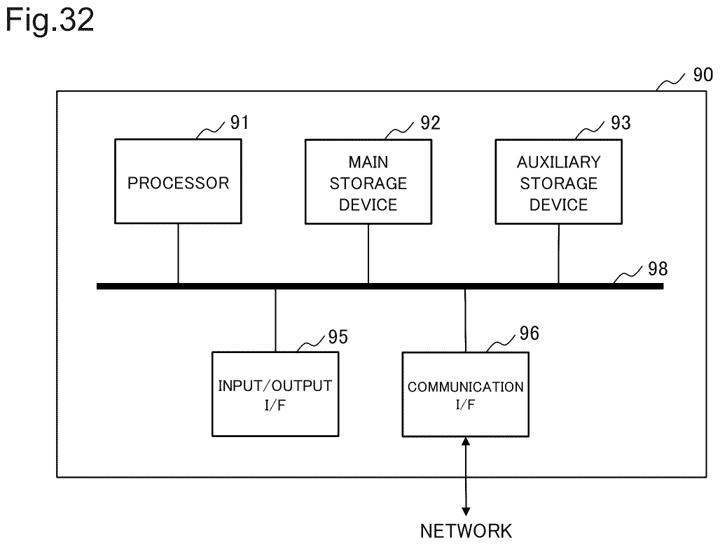

Here, a hardware configuration for executing control and processing according to each example embodiment of the present disclosure will be described using an information processing device 90 of FIG. 32 as an example. The information processing device 90 in FIG. 32 is a configuration example for executing control and processing of each example embodiment, and does not limit the scope of the present disclosure.

As illustrated in FIG. 32, the information processing device 90 includes a processor 91, a main storage device 92, an auxiliary storage device 93, an input/output interface 95, and a communication interface 96. In FIG. 32, the interface is abbreviated as I/F. The processor 91, the main storage device 92, the auxiliary storage device 93, the input/output interface 95, and the communication interface 96 are connected to be capable of data communication via a bus 98. The processor 91, the main storage device 92, the auxiliary storage device 93, and the input/output interface 95 are connected to a network such as the Internet or an intranet via the communication interface 96.

The processor 91 loads a program stored in the auxiliary storage device 93 or the like to the main storage device 92. The processor 91 executes the program loaded on the main storage device 92. In the present example embodiment, it is sufficient if a software program installed in the information processing device 90 is used. The processor 91 executes control and processing according to each example embodiment.

The main storage device 92 has an area in which a program is loaded. A program stored in the auxiliary storage device 93 or the like is loaded to the main storage device 92 by the processor 91. The main storage device 92 is achieved by, for example, a volatile memory such as dynamic random access memory (DRAM). A nonvolatile memory such as magnetoresistive random access memory (MRAM) may be configured/added as the main storage device 92.

The auxiliary storage device 93 stores various data such as a program. The auxiliary storage device 93 is achieved by a local disk such as a hard disk or flash memory. Various data may be stored in the main storage device 92, and the auxiliary storage device 93 may be omitted.

The input/output interface 95 is an interface for connecting the information processing device 90 and a peripheral device based on standards or specifications. The communication interface 96 is an interface for connecting to an external system or device through a network such as the Internet or an intranet based on standards or specifications. The input/output interface 95 and the communication interface 96 may be shared as an interface connected to an external device.

Input devices such as a keyboard, a mouse, and a touch panel may be connected to the information processing device 90 as necessary. These input devices are used to input information and settings. When the touch panel is used as the input device, the display screen of the display device may be configured to serve as the interface of the input device. It is sufficient if data communication between the processor 91 and the input device is mediated by the input/output interface 95.

The information processing device 90 may be provided with a display device for displaying information. In a case where a display device is provided, the information processing device 90 preferably includes a display control device (not illustrated) for controlling display of the display device. It is sufficient if the display device is connected to the information processing device 90 via the input/output interface 95.

The information processing device 90 may be provided with a drive device. The drive device mediates reading of data and a program from a recording medium, writing of a processing result of the information processing device 90 to the recording medium, and the like between the processor 91 and the recording medium (program recording medium). It is sufficient if the drive device is connected to the information processing device 90 via the input/output interface 95.

The above is an example of a hardware configuration for enabling control and processing according to each example embodiment of the present invention. The hardware configuration in FIG. 32 is an example of the hardware configuration for executing control and processing of each example embodiment, and does not limit the scope of the present invention. A program for causing a computer to execute control and processing according to each example embodiment is also included in the scope of the present invention. Further, a program recording medium in which the program according to each example embodiment is recorded is also included in the scope of the present invention. The recording medium can be achieved by, for example, an optical recording medium such as a compact disc (CD) or a digital versatile disc (DVD). The recording medium may be achieved by a semiconductor recording medium such as a universal serial bus (USB) memory or a secure digital (SD) card. The recording medium may be achieved by a magnetic recording medium such as a flexible disk, or another recording medium. When a program executed by the processor is recorded in a recording medium, the recording medium corresponds to a program recording medium.

The components of each example embodiment may be combined in any manner. The components of each example embodiment may be achieved by software or may be achieved by a circuit.

While the invention has been particularly shown and described with reference to exemplary embodiments thereof, the invention is not limited to these embodiments. It will be understood by those of ordinary skill in the art that various changes in form and details may be made therein without departing from the spirit and scope of the present invention as defined by the claims.

Some or all of the above example embodiments may also be described as the following supplementary notes, but are not limited to the following.

### (Supplementary Note 1)

A fall probability estimation device including:
a data acquisition unit that acquires feature amount data including a feature amount extracted from sensor data regarding a motion of a foot of a user and used to estimate fall probability of the user;
a storage unit that stores an estimation model that outputs a fall probability index according to an input of the feature amount data;
an estimation unit that inputs the acquired feature amount data into the estimation model and estimates the fall probability of the user according to the fall probability index output from the estimation model; and
an output unit that outputs information regarding the estimated fall probability of the user.

### (Supplementary Note 2)

The fall probability estimation device according to supplementary note 1, in which
the data acquisition unit
acquires the feature amount data including a feature amount extracted from gait waveform data generated using time-series data of the sensor data regarding a motion of a foot and used to estimate a fall probability score as the fall probability index,
the storage unit
stores the estimation model that outputs the fall probability score according to an input of the feature amount data, and
the estimation unit
inputs the acquired feature amount data to the estimation model, and estimates the fall probability of the user according to the fall probability score output from the estimation model.

### (Supplementary Note 3)

The fall probability estimation device according to supplementary note 2, in which
the storage unit
stores the estimation model generated by machine learning using training data regarding a plurality of subjects in which a feature amount used for estimation of the fall probability index is an explanatory variable and the fall probability index of the plurality of subjects is an objective variable, and
the estimation unit
inputs the feature amount data acquired regarding the user to the estimation model, and estimates the fall probability of the user according to the fall probability index of the user output from the estimation model.

### (Supplementary Note 4)

The fall probability estimation device according to supplementary note 3, in which
the storage unit
stores the estimation model machine-learned using an explanatory variable including attribute data of the plurality of subjects, and
the estimation unit
inputs the feature amount data and the attribute data related to the user to the estimation model, and estimates the fall probability of the user according to the fall probability index of the user output from the estimation model.

### (Supplementary Note 5)

The fall probability estimation device according to supplementary note 3 or 4, in which
the storage unit
stores a first estimation model generated by machine learning using training data regarding the gait waveform data of the plurality of subjects in which feature amounts regarding activities of quadriceps femoris, hamstrings, tibialis anterior muscle, tibialis posterior muscle, and gluteus medius muscle extracted from a first section from immediately before heel contact to immediately after landing of a sole, feature amounts regarding activities of iliopsoas muscle, quadriceps femoris, adductor longus muscle, gracilis muscle, sartorius muscle, and tibialis anterior muscle extracted from a second section from a pre-swing period to an initial swing period, and feature amounts regarding activities of hamstrings, tibialis anterior muscle, and gracilis muscle extracted from a third section before and after movement of minimum toe clearance in a mid-swing period are explanatory variables, and the fall probability index of the plurality of subjects is an objective variable, and
the estimation unit
inputs the feature amount data acquired according to gait of the user to the first estimation model, and estimates the fall probability of the user according to the fall probability index of the user output from the first estimation model.

### (Supplementary Note 6)

The fall probability estimation device according to supplementary note 5, in which
the storage unit
stores the first estimation model generated by machine learning using training data regarding the gait waveform data of the plurality of subjects in which at least one feature amount included in a first feature amount group including at least one feature amount extracted from the first section, a second feature amount group including at least one feature amount extracted from the second section, and a third feature amount group including at least one feature amount extracted from the third section is an explanatory variable and the fall probability index of the plurality of subjects is an objective variable,
the data acquisition unit
acquires the feature amount data including the at least one feature amount included in the first feature amount group, the second feature amount group, and the third feature amount group extracted according to the gait of the user, and
the estimation unit
inputs the acquired feature amount data to the first estimation model, and estimates the fall probability of the user according to the fall probability index of the user output from the first estimation model.

### (Supplementary Note 7)

The fall probability estimation device according to supplementary note 6, in which
the storage unit
stores the first estimation model generated by machine learning using training data regarding the gait waveform data of the plurality of subjects in which at least one feature amount extracted from a gait phase common to five items among gait phases from which feature amounts regarding each of the five items are extracted: a total muscular strength of a whole body; a dynamic balance; a lower-limb muscular strength: a mobility; and a static balance is an explanatory variable and the fall probability index of the plurality of subjects is an objective variable,
the data acquisition unit
acquires the feature amount data including the at least one feature amount included in the first feature amount group, the second feature amount group, and the third feature amount group extracted according to the gait of the user, and
the estimation unit
inputs the acquired feature amount data to the first estimation model, and estimates the fall probability of the user according to the fall probability index of the user output from the first estimation model.

### (Supplementary Note 8)

The fall probability estimation device according to supplementary note 3 or 4, in which
the storage unit
stores a second estimation model generated by machine learning using training data in which a score of at least one of five items: a total muscular strength of a whole body; a dynamic balance; a lower-limb muscular strength; a mobility; and a static balance estimated using the gait waveform data of the plurality of subjects is an explanatory variable and the fall probability index of the plurality of subjects is an objective variable,
the data acquisition unit
acquires at least one of the scores regarding the five items estimated according to gait of the user, and
the estimation unit
inputs the acquired score to the second estimation model, and estimates the fall probability of the user according to the fall probability index of the user output from the second estimation model.

### (Supplementary Note 9)

The fall probability estimation device according to supplementary note 8, in which
the storage unit
stores a pre-estimation model generated by machine learning using training data regarding the gait waveform data of the plurality of subjects in which a feature amount related to at least one of the five items is an explanatory variable and the scores of the five items related to the feature amount used as the explanatory variable are objective variables,
the data acquisition unit
acquires a feature amount extracted according to the gait of the user and related to at least one of the five items, and
the estimation unit
inputs the acquired feature amount to the pre-estimation model, inputs the score output from the pre-estimation model to the second estimation model, and estimates the fall probability of the user according to the fall probability index of the user output from the second estimation model.

### (Supplementary Note 10)

A fall probability estimation system including:
the fall probability estimation device according to any one of supplementary notes 1 to 9; and
a gait measurement device including a sensor that is installed in footwear of a user who is an estimation target of fall probability, measures a spatial acceleration and a spatial angular velocity, generates sensor data regarding a motion of a foot by using the measured spatial acceleration and spatial angular velocity, and outputs the generated sensor data, and a feature amount data generation unit that acquires time-series data of the sensor data including a feature of gait, extracts gait waveform data for one gait cycle from the time-series data of the sensor data, normalizes the extracted gait waveform data, extracts a feature amount used for estimation of the fall probability from a gait phase cluster including at least one temporally continuous gait phase from the normalized gait waveform data, generates feature amount data including the extracted feature amount, and outputs the generated feature amount data to the fall probability estimation device.

### (Supplementary Note 11)

The fall probability estimation system according to supplementary note 10, in which
the fall probability estimation device is
mounted on a terminal device having a screen visually recognizable by the user, and
displays information regarding the fall probability estimated according to the feature amount extracted from the sensor data regarding the motion of the foot of the user on the screen of the terminal device.

### (Supplementary Note 12)

The fall probability estimation system according to supplementary note 11, in which
the fall probability estimation device
displays recommendation information according to the fall probability estimated according to the feature amount extracted from the sensor data regarding the motion of the foot of the user on the screen of the terminal device.

### (Supplementary Note 13)

The fall probability estimation system according to supplementary note 12, in which
the fall probability estimation device
displays, on the screen of the terminal device, a video related to training for building up a body part related to the fall probability as the recommendation information according to the fall probability estimated according to the feature amount extracted from the sensor data regarding the motion of the foot of the user.

### (Supplementary Note 14)

A fall probability estimation method including:
by a computer
acquiring feature amount data including a feature amount extracted from sensor data regarding a motion of a foot of a user and used for estimation of fall probability of the user;
inputting the acquired feature amount data to an estimation model that outputs a fall probability index according to an input of the feature amount data;
estimating the fall probability of the user according to the fall probability index output from the estimation model; and
outputting information regarding the estimated fall probability of the user.

### (Supplementary Note 15)

A program causing a computer to execute:
processing of acquiring feature amount data including a feature amount extracted from sensor data regarding a motion of a foot of a user and used for estimation of fall probability of the user;
processing of inputting the acquired feature amount data to an estimation model that outputs a fall probability index according to an input of the feature amount data;
processing of estimating the fall probability of the user according to the fall probability index output from the estimation model; and
processing of outputting information regarding the estimated fall probability of the user.

### REFERENCE SIGNS LIST

1 fall probability estimation system
2 machine learning system
10, 20 gait measurement device
11 sensor
12 feature amount data generation unit
13 fall probability estimation device
25 machine learning device
111 acceleration sensor
112 angular velocity sensor
121 acquisition unit
122 normalization unit
123 extraction unit
125 generation unit
127 feature amount data output unit
131, 331 data acquisition unit
132, 332 storage unit
133, 333 estimation unit
135, 335 output unit
251 reception unit
253 machine learning unit
255 storage unit

What is claimed is:

1. A fall probability estimation device comprising:
a storage configured to store an estimation model that outputs a fall probability index according to an input of feature amount data used for estimating fall probability;
a memory storing instructions; and
a processor connected to the memory and configured to execute the instructions to:
acquire feature amount data including a feature amount extracted from sensor data regarding a motion of a foot of a user and used for estimation of fall probability of the user;
input the acquired feature amount data into the estimation model and estimate the fall probability of the user according to the fall probability index output from the estimation model; and
output information regarding the estimated fall probability of the user.

2. The fall probability estimation device according to claim 1, wherein
the storage stores the estimation model that outputs a fall probability score according to an input of the feature amount data, and
the processor is configured to execute the instructions to
acquire the feature amount data including a feature amount extracted from gait waveform data generated using time-series data of the sensor data regarding a motion of a foot and used to estimate the fall probability score as the fall probability index,
input the acquired feature amount data to the estimation model, and
estimate the fall probability of the user according to the fall probability score output from the estimation model.

3. The fall probability estimation device according to claim 2, wherein
the storage stores the estimation model generated by machine learning using training data regarding a plurality of subjects in which a feature amount used for estimation of the fall probability index is an explanatory variable and the fall probability index of the plurality of subjects is an objective variable, and
the processor is configured to execute the instructions to
input the feature amount data acquired regarding the user to the estimation model, and
estimate the fall probability of the user according to the fall probability index of the user output from the estimation model.

4. The fall probability estimation device according to claim 3, wherein
the storage stores the estimation model machine-learned using an explanatory variable including attribute data of the plurality of subjects, and
the processor is configured to execute the instructions to
input the feature amount data and the attribute data related to the user to the estimation model, and
estimate the fall probability of the user according to the fall probability index of the user output from the estimation model.

5. The fall probability estimation device according to claim 3, wherein
the storage stores a first estimation model generated by machine learning using training data regarding the gait waveform data of the plurality of subjects in which feature amounts regarding activities of quadriceps femoris, hamstrings, tibialis anterior muscle, tibialis posterior muscle, and gluteus medius muscle extracted from a first section from immediately before heel contact to immediately after landing of a sole, feature amounts regarding activities of iliopsoas muscle, quadriceps femoris, adductor longus muscle, gracilis muscle, sartorius muscle, and tibialis anterior muscle extracted from a second section from a pre-swing period to an initial swing period, and feature amounts regarding activities of hamstrings, tibialis anterior muscle, and gracilis muscle extracted from a third section before and after movement of minimum toe clearance in a mid-swing period are explanatory variables, and the fall probability index of the plurality of subjects is an objective variable, and
the processor is configured to execute the instructions to
input the feature amount data acquired according to gait of the user to the first estimation model, and
estimate the fall probability of the user according to the fall probability index of the user output from the first estimation model.

6. The fall probability estimation device according to claim 5, wherein
the storage stores the first estimation model generated by machine learning using training data regarding the gait waveform data of the plurality of subjects in which at least one feature amount included in a first feature amount group including at least one feature amount extracted from the first section, a second feature amount group including at least one feature amount extracted from the second section, and a third feature amount group including at least one feature amount extracted from the third section is an explanatory variable and the fall probability index of the plurality of subjects is an objective variable,
the processor is configured to execute the instructions to
acquire the feature amount data including the at least one feature amount included in the first feature amount group, the second feature amount group, and the third feature amount group extracted according to the gait of the user,
input the acquired feature amount data to the first estimation model, and
estimate the fall probability of the user according to the fall probability index of the user output from the first estimation model.

7. The fall probability estimation device according to claim 6, wherein
the storage stores the first estimation model generated by machine learning using training data regarding the gait waveform data of the plurality of subjects in which at least one feature amount extracted from a gait phase common to five items among gait phases from which feature amounts regarding each of the five items are extracted: a total muscular strength of a whole body; a dynamic balance; a lower-limb muscular strength; a mobility; and a static balance is an explanatory variable and the fall probability index of the plurality of subjects is an objective variable,
the processor is configured to execute the instructions to
acquire the feature amount data including the at least one feature amount included in the first feature amount group, the second feature amount group, and the third feature amount group extracted according to the gait of the user,
input the acquired feature amount data to the first estimation model, and
estimate the fall probability of the user according to the fall probability index of the user output from the first estimation model.

8. The fall probability estimation device according to claim 3, wherein
the storage stores a second estimation model generated by machine learning using training data in which a score of at least one of five items: a total muscular strength of a whole body; a dynamic balance; a lower-limb muscular strength; a mobility; and a static balance estimated using the gait waveform data of the plurality of subjects is an explanatory variable and the fall probability index of the plurality of subjects is an objective variable,
the processor is configured to execute the instructions to
acquire at least one of the scores regarding the five items estimated according to gait of the user,
input the acquired score to the second estimation model, and
estimate the fall probability of the user according to the fall probability index of the user output from the second estimation model.

9. The fall probability estimation device according to claim 8, wherein
the storage stores a pre-estimation model generated by machine learning using training data regarding the gait waveform data of the plurality of subjects in which a feature amount related to at least one of the five items is an explanatory variable and the scores of the five items related to the feature amount used as the explanatory variable are objective variables,
the processor is configured to execute the instructions to
acquire a feature amount extracted according to the gait of the user and related to at least one of the five items,
input the acquired feature amount to the pre-estimation model,
input the score output from the pre-estimation model to the second estimation model, and
estimate the fall probability of the user according to the fall probability index of the user output from the second estimation model.

10. A fall probability estimation system comprising:
the fall probability estimation device according to any one of claim 1; and
a gait measurement device including
a sensor that is installed in footwear of a user who is an estimation target of fall probability, measures a spatial acceleration and a spatial angular velocity, generates sensor data regarding a motion of a foot by using the measured spatial acceleration and spatial angular velocity, and outputs the generated sensor data,
a memory storing instructions, and
a processor connected to the memory and configured to execute the instructions to
acquire time-series data of the sensor data including a feature of gait,
extract gait waveform data for one gait cycle from the time-series data of the sensor data,
normalize the extracted gait waveform data,
extract a feature amount used for estimation of the fall probability from a gait phase cluster including at least one temporally continuous gait phase from the normalized gait waveform data,
generate feature amount data including the extracted feature amount, and
output the generated feature amount data to the fall probability estimation device.

11. The fall probability estimation system according to claim 10, wherein
the fall probability estimation device is mounted on a terminal device having a screen visually recognizable by the user, and
the processer of the fall probability estimation device is configured to execute the instructions to display information regarding the fall probability estimated according to the feature amount extracted from the sensor data regarding the motion of the foot of the user on the screen of the terminal device.

12. The fall probability estimation system according to claim 11, wherein
the processer of the fall probability estimation device is configured to execute the instructions to
display recommendation information according to the fall probability estimated according to the feature amount extracted from the sensor data regarding the motion of the foot of the user on the screen of the terminal device.

13. The fall probability estimation system according to claim 12, wherein
the processer of the fall probability estimation device is configured to execute the instructions to
display, on the screen of the terminal device, a video related to training for building up a body part related to the fall probability as the recommendation information according to the fall probability estimated according to the feature amount extracted from the sensor data regarding the motion of the foot of the user.

14. A fall probability estimation method executed by a computer, the method comprising:
acquiring feature amount data including a feature amount extracted from sensor data regarding a motion of a foot of a user and used for estimation of fall probability of the user;
inputting the acquired feature amount data to an estimation model that outputs a fall probability index according to an input of the feature amount data;
estimating the fall probability of the user according to the fall probability index output from the estimation model; and
outputting information regarding the estimated fall probability of the user.

15. A non-transitory recording medium recording a program causing a computer to execute:
processing of acquiring feature amount data including a feature amount extracted from sensor data regarding a motion of a foot of a user and used for estimation of fall probability of the user;
processing of inputting the acquired feature amount data to an estimation model that outputs a fall probability index according to an input of the feature amount data;
processing of estimating the fall probability of the user according to the fall probability index output from the estimation model; and
processing of outputting information regarding the estimated fall probability of the user.

16. The static balance estimation system according to claim 12, wherein
the processor of the mobility estimation device is configured to execute the instructions to
cause the recommendation information that supports the user for making decision about taking an action to be displayed on the screen of the terminal device.
